SEQUENCE LISTING (10 sequences)

SEQ ID NO: 1 - DNA, 20 bp, Artificial Sequence
  synthetic nucleic acid
ggacctcatggcctacatgg

SEQ ID NO: 2 - DNA, 18 bp, Artificial Sequence
  synthetic nucleic acid
tagggcctctcttgctca

SEQ ID NO: 3 - DNA, 19 bp, Artificial Sequence
  synthetic nucleic acid
gtcggagtcaacggatttg

SEQ ID NO: 4 - DNA, 22 bp, Artificial Sequence
  synthetic nucleic acid
ccatgtagttgaggtcaatgaa

SEQ ID NO: 5 - DNA, 20 bp, Artificial Sequence
  synthetic nucleic acid
aacccacgatcaactgaagc

SEQ ID NO: 6 - DNA, 20 bp, Artificial Sequence
  synthetic nucleic acid
ttgaggctgttgcttgtgtg

SEQ ID NO: 7 - DNA, 18 bp, Artificial Sequence
  synthetic nucleic acid
ctatctcgcacctgaaac

SEQ ID NO: 8 - DNA, 19 bp, Artificial Sequence
  synthetic nucleic acid
gagggtggatggaattaag

SEQ ID NO: 9 - protein, 11 aa, Artificial Sequence
  synthetic polypeptide
YGRKKRRQRRR

SEQ ID NO: 10 - protein, 7 aa, Artificial Sequence
  synthetic polypeptide
DLLPRGS

(19) United States
(12) Patent Application Publication
(10) Pub. No.: US 2019/0212344 A1
(43) Pub. Date: Jul. 11, 2019

(54) BIOMARKERS FOR USE IN DETERMINING RESPONSE TO TREATMENT OF NEURODEGENERATION DISEASE

(71) Applicant: The Board of Trustees of the Leland Stanford Junior University, Stanford, CA (US)

(72) Inventors: Marie-Helene Dziesietnik, Palo Alto, CA (US); Daria Mochly-Rosen, Menlo Park, CA (US)

(21) Appl. No.: 16/333,979

(22) PCT Filed: Sep. 14, 2017
(86) PCT No.: PCT/US2017/051520

§ 371 Date: Mar. 15, 2019

Related U.S. Application Data
(60) Provisional application No. 62/395,225, filed on Sep. 15, 2016.

(51) Int. Cl. G01N 33/68; C12Q 1/6883; A61K 38/08; A61P 25/28

(52) U.S. Cl. CPC G01N 33/6896; C12Q 1/6883; C12Q 2600/118; A61P 25/28; C12Q 2600/106; A61K 38/08

(57) ABSTRACT

The diagnosis of a neurodegenerative disease or the response of a patient with a neurodegenerative disease to therapy, in a clinical trial setting or in a long-term disease management setting, is assessed.

### CROSS-REFERENCE TO RELATED APPLICATION

This application claims the benefit of U.S. Provisional Application No. 62/395,225, filed Sep. 15, 2016, herein specifically incorporated by reference.

### BACKGROUND

Huntington's disease (HD) is a fatal autosomal-dominant neurodegenerative disease caused by an expanded trinucleotide CAG repeat in the gene encoding the huntingtin protein. HD is a progressive disease that affects middle age carriers, and the severity of the disease correlates with the length of the CAG repeat. Patients affected by HD display a loss of neurons predominantly in the striatum and cortex that is progressively accompanied by a loss of voluntary and involuntary movements as well as psychiatric and cognitive disturbances. Patients usually die 10-15 years after the onset of the disease due to immobility-induced complications. Currently, there is no cure for the disease and no treatment effectively slows down the disease progression.

The neurological symptoms of HD are due to the aggregation of the mutant huntingtin (mtHtt) protein in neurons that causes, among other pathologies, mitochondrial dysfunction. This, in turn, leads to loss of ATP and an increase in oxidative stress. Evidence from studies in human HD subjects and experimental HD mouse models suggests that mitochondrial dysfunction precedes neuropathology and clinical symptoms, indicating that mitochondrial impairment is an early event in the cascade of events leading to HD pathology.

Proper mitochondrial function is maintained, in part, by balanced mitochondrial dynamics, i.e., a balance between an increase in mitochondrial number by fission and a decrease in mitochondrial number by fusion. A defect in either fusion or fission limits mitochondrial motility, decreases energy production and increases oxidative stress, thereby promoting cell dysfunction and death. The two opposing processes, fusion and fission, are controlled by evolutionarily conserved large GTPases that belong to the dynamin family of proteins. In mammalian cells, mitochondrial fusion is regulated by mitofusin-1 and -2 (MFN-1/2) and optic atrophy 1 (OPA1), whereas mitochondrial fission is controlled by dynamin-1-related protein, Drp1.

Drp1 is primarily found in the cytosol, but it translocates from the cytosol to the mitochondrial surface in response to various cellular stimuli to regulate mitochondrial morphology. At the mitochondrial surface, Drp1 is thought to wrap around the mitochondria to induce fission powered by its GTPase activity. The association of Drp1 with the mitochondrial outer membrane and its activity in mammalian cells depends on various accessory proteins. Fis1 is an integral mitochondrial outer membrane protein that recruits Drp1 to promote fission. A selective peptide inhibitor of Drp1 and consequent pathological mitochondrial fragmentation, P110, has been identified and developed in a strategy to inhibit mtHtt-induced neurotoxicity (for example, see Guo et al. (2013) J. Clin. Invest. 123(12):5371; the entire content of which is incorporated herein by reference).

HD patients can unequivocally be diagnosed via genetic testing for expansion of CAG trinucleotide repeats in the HTT gene. The challenge, then, is how a response to treatment can be assessed. Furthermore, changes in affected individuals must occur from the time of conception, yet neurodegeneration symptoms are not apparent for more than 40 or 50 years. Therefore, although ideally, therapeutic interventions should begin in pre-symptomatic subjects, it is prohibitively expensive to await several decades to assess the benefit of that intervention. A candidate biomarker should show a measurable response to the progression and severity of the disease.

There is an urgent need for the development of sensitive, specific and non-invasive biomarkers for assessing drug efficacy in the treatment of patients with neurodegenerative diseases. The ideal biomarker would not only facilitate clinical trials of drug candidates, but would also find utility in disease management of patients who are prescribed such medications. The present invention addresses this emerging but unmet medical need.

### SUMMARY

Methods are provided for clinical monitoring of the treatment of neurodegenerative disease, which diseases include, without limitation, Parkinson's disease, Huntington's disease, Alzheimer's disease, amyotrophic lateral sclerosis, ischemic neuronal damage, diabetes-induced neuropathy and the like. The treatment may be in a clinical trial format, or may track efficacy of treatment of an individual.

Although genetic testing readily identifies those who are or will be affected by HD, current pharmacological treatments do not prevent or slow down disease progression. Challenges in developing HD treatment arise from the slow progression of clinical symptoms and the inability to biopsy the affected tissue—the brain, thus making clinical trials to assess treatment benefit long and very expensive. Human trials in HD are, moreover, time-consuming due to the slow progression of the disease, its insidious onset and patient-to-patient variability. There is also a need to include a large cohort of patients, because many of the clinical assessments are quite subjective (e.g., psychiatric tests), and the inability to biopsy the affected tissue—neurons from living patients in the brain

In some embodiments the neurodegenerative disease is Huntington's disease. In some embodiments treatment of an individual is provided in accordance with the results of the clinical monitoring analysis. Biomarkers are identified that could be used as surrogate markers to determine the benefit of therapeutic intervention to prevent or delay the onset of the disease in diagnosed but asymptomatic HD patients, or to reduce disease symptoms in symptomatic HD patients. Levels of these biomarkers can be positively correlated with the improvement seen with therapeutic intervention.

In some embodiments an individual is treated with a therapeutic agent or regimen, and the effectiveness of treatment is determined by analysis of one or more peripheral biomarkers described herein. A treatment that is successful for an individual as evidenced by changes in biomarkers described herein is continued for the individual, or continued in the context of, for example, a clinical trial, for a plurality of individuals. A treatment that is not successful as evidenced by changes in biomarkers described herein is discontinued for the individual, or discontinued in the context of, for example, a clinical trial, for a plurality of individuals.

A benefit of the biomarkers described herein is that they are detectable in peripheral tissues, and thus provide surrogate markers that are indicators for the progression and treatment of disease in the brain since increases in levels of these surrogate markers are correlated with disease progression and decreases in levels of these surrogate markers are correlated with efficacious treatment of disease in the brain. Peripheral biomarkers to assist in clinical monitoring of neurodegenerative disease include markers related to (i) mitochondrial and cell integrity, e.g. measuring mitochondrial DNA in the plasma; (ii) mtHtt aggregation in the peripheral tissue; and (iii) evidence of increased oxidative stress, e.g. as measured by increased levels of 4-hydroxynonenal (4-HNE) adducts and DNA damage, including without limitation detecting the presence of 8-hydroxy-deoxy-guanosine (8-OHdG) in urine or plasma samples. The levels of these biomarkers are normalized i.e. the level is changed to a level closer to that of a normal, non-diseased biomarker. In some embodiment the level of a biomarker is reduced and is closer to a non-disease level by an effective treatment that also reduces the symptoms and pathology in subjects afflicted with HD, animal models for HD, etc. Methods of the invention may measure at least one peripheral biomarker, at least two peripheral biomarkers, at least 3 peripheral biomarkers, or more. In some embodiments where two or more biomarkers are measured, each biomarker is selected from a different class, i.e. (i) mitochondrial and cell integrity; (ii) mtHtt aggregation in the peripheral tissue; and (iii) evidence of increased oxidative stress.

In some embodiments of the invention the treatment comprises administration of an inhibitor of mitochondrial fission. In one embodiment, the fission inhibitor inhibits GTPase activity of a Drp1 polypeptide. In another embodiment, the fission inhibitor selectively inhibits GTPase activity of a Drp1 polypeptide. The fission inhibitor may be a peptide, e.g. P110; or a peptide comprising P110; or a genetic construct encoding P110. In one embodiment, the fission inhibitor inhibits binding of a Fis1 polypeptide to a Drp1 polypeptide. In another embodiment, the fission inhibitor selectively inhibits binding of a Fis1 polypeptide to a Drp1 polypeptide. In one embodiment, the fission inhibitor reduces or inhibits mitochondrial fragmentation in a cell. In another embodiment, the fission inhibitor reduces or inhibits fragmentation in a cell which has been stressed.

In some embodiments the methods and biomarkers provided herein are utilized for monitoring ongoing therapeutic regimens for neurodegenerative diseases. In other embodiments, the methods of the invention are used in determining the efficacy of a therapy for treatment of a neurodegenerative disease, either at an individual level, or in the analysis of a group of patients, e.g. in a clinical trial format. In another embodiment methods of the invention are used to determine appropriate timing for initiation of therapeutic intervention. Such embodiments typically involve the comparison of two or more time points for a patient or group of patients. The patient status is expected to differ between the two time points as the result of administration of a therapeutic agent or regimen.

In some embodiments, a patient sample is obtained prior to treatment, as a control, and compared to samples from the same patient following treatment. In other embodiments, the biomarkers of mitochondrial function are assessed over long periods of time to monitor patient status. One or more of urine; plasma; and skin or muscle tissue samples may be collected for analysis at one or more timepoints, such as two or more timepoints, e.g. at 3 time points, 4, 5, 6, 7 or more, and may be monitored at regular intervals during the course of treatment.

In some embodiments, the level of mitochondrial DNA in plasma is measured as a marker for therapeutic efficacy for treatment of neurodegenerative disease. In some embodiments, the mitochondrial DNA is cytochrome C oxidase. In some embodiments the mitochondrial DNA is mtND2 (mitochondria encoded NADH dehydrogenase 2; a subunit of complex 1 located at the inner mitochondrial membrane. In some embodiments, the measuring is performed with quantitative PCR. It is shown herein that levels of mitochondrial DNA in plasma initially rise, prior to overt neurological symptoms, and then drop during clinical stages of disease, e.g. a decrease of up to about 10% relative to a normal control, a decrease of up to about 20% relative to a normal control, a decrease of up to about 30% relative to a normal control, a decrease of up to about 40% relative to a normal control, a decrease of up to about 50% relative to a normal control, or more.

Effective treatment normalizes levels of mitochondrial DNA in plasma. In a patient where there has been a drop in mtDNA is plasma, for example a patient showing clinical signs of disease, treatment may provide for an increase relative to pre-treatment levels, and may be an increase of up to about 10%, up to about 20%, up to about 30%, up to about 40%, up to about 50%, or more, and may include an increase to a level substantially the same as a normal control, where a normal control may be an individual without the disease and without a predisposition to the disease.

In patients where mtDNA is increased relative to a normal control, which include without limitation patients treated in early or asymptomatic stages of disease, effective therapy normalizing towards control values will decrease levels of plasma mtDNA relative to pre-treatment values. Therapy may provide for a decrease of up to about 10%, up to about 20%, up to about 30%, up to about 40%, up to about 50%, or more, and may include a decrease to a level substantially the same as a normal control, where a normal control may be an individual without the disease and without a predisposition to the disease.

Maximal neuronal loss occurs earlier than motor and behavioral impairments, and is evidenced by an initial increase in mtDNA in plasma, preceding maximal behavioral deficits, consistent with the evidence that mitochondrial damage occurred at the early stage of disease. Assessing mtDNA in the plasma provides a useful marker to indicate early HD-associated pathology and response to therapy.

In some embodiments a plasma sample, alone or in combination with analysis of mtDNA, is measured for levels of inflammatory cytokines, including without limitation IL-6, TNFα, etc. During the course of disease, plasma concentrations of inflammatory cytokines may increase by at least about 30%, at least about 50%, at least about 1-fold, at least about 2-fold or more relative to a normal control. Successful treatment with a therapeutic agent or regimen normalizes levels, e.g. a decrease of up to about 10% relative to pre-treatment levels, a decrease of up to about 20%, a decrease of up to about 30%, a decrease of up to about 40%, a decrease of up to about 50%, or more, and may include a decrease to a level substantially the same as a normal control.

In some embodiments a urine sample is analyzed for the level of oxidative DNA damage products, including without limitation 8-OHdG, which is a product of guanine oxidation by oxidative stress that is found in the urine as a product of DNA excision repair. The measuring may be performed, e.g. by ELISA, mass spectroscopy, etc. During the course of disease, urine concentrations of oxidative DNA damage products, including without limitation 8-OHdG, may increase by at least about 30%, at least about 50%, at least about 1-fold, at least about 2-fold, at least about 3-fold, or more relative to a normal control. Successful treatment with a therapeutic agent or regimen normalizes levels, e.g. a decrease of up to about 10% relative to pre-treatment levels, a decrease of up to about 20%, a decrease of up to about 30%, a decrease of up to about 40%, a decrease of up to about 50%, or more, and may include a decrease to a level substantially the same as a normal control.

In some embodiments, where the individual is a HD patient, a peripheral tissue sample, e.g. a skin biopsy sample, a muscle biopsy sample, etc. is analyzed for levels of mtHtt aggregation; protein oxidation markers, for example 4-HNE adducts; and the like. As monitored by immunohistochemistry, there is an increase in mtHtt aggregates at the periphery of the muscle fibers and skin section relative to a normal control. Successful treatment reduces these aggregates, and reduces protein oxidation markers. Immunohistochemistry can be performed, for example, with antibodies specific for mtHtt; antibodies specific for 4-HNE, etc.

In an embodiment, the method is implemented by computer. In an embodiment, the method further comprises selecting a therapeutic regimen based on the analysis. In an embodiment, the method further comprises determining a treatment course for the subject based on the analysis.

### BRIEF DESCRIPTION OF THE DRAWINGS

The invention is best understood from the following detailed description when read in conjunction with the accompanying drawings. The patent or application file contains at least one drawing executed in color. It is emphasized that, according to common practice, the various features of the drawings are not to-scale. On the contrary, the dimensions of the various features are arbitrarily expanded or reduced for clarity. Included in the drawings are the following figures.

FIG. 1: Analysis of mitochondrial DNA content in brain and plasma. (A) DNA synthesized from mouse brain tissue was used in real-time PCR with mtND2 primers targeting mouse mtDNA (n=4 mice per group). (B) mtND2 DNA content in plasma samples from mouse blood collected at the age of 13 weeks were assayed in real-time PCR (n=10 WT, n=8 Tg). Housekeeping nuclear gene, GAPDH, was used for normalization. (C) mtND2 levels over time in mice. Mouse plasma samples from WT and R6/2 mice were collected every 2 weeks and mtND2 levels was determined using real-time PCR. mtND2 levels decreased overtime in R6/2 mice compared to WT mice (n=10 WT; n=10 R6/2 (5-11 weeks); n=9 R6/2 at 13 weeks). The data are presented as mean±SEM of 2^{-ΔΔC}T.

FIG. 2: Behavioral phenotype of R6/2 mice compared to WT. (A) R6/2 mice have significantly decreased latency to fall during the accelerating Rotor Rod test (4-40 rpm) starting at 7 weeks of age and progressively shorter latency as they age. WT mice were able to maintain their latency to fall throughout the test (p=0.0001 WT vs R6/2). (B) 11 weeks old WT mice were able to find the new escape box location during DMP dry maze test and their latency to find the escape box decreased as more training days were conducted. The latency to find the escape box for R6/2 mice at 11 weeks old remain stagnant during the 4 trials of training per day and across multiple days of testing (p=0.0001 WT vs R6/2).

FIG. 3: Beneficial effect of P110 on mtND2 levels in R6/2 plasma. (A) WT and R6/2 mice were treated with control TAT or with P110 for 8 weeks (n=6/group WT TAT and P110; n=5/group R6/2 TAT and P110). (B) WT and R6/2 mice were treated with control TAT or with P110 for 1 week followed by 3 weeks no treatment before another week of treatment was administered. Mice plasma samples were collected 3 weeks after the end of the treatment, at the age of 13 weeks. (n=9/group WT TAT and P110; n=11 R6/2 TAT; n=15 R6/2 P110). The data are presented as mean±SEM of 2^{-ΔΔC}T. (C) Survival curve of WT and R6/2 mice treated with P110 as in (A). 4 mice out of 10 R6/2 TAT-treated mice and 2 mice out of 10 R6/2 P110-treated mice died at the age of 13 weeks. The results are shown as log-Rank (mantel-cox) test, chi square=10.9, p=0.0123). (D) The effect of intermittent treatment of P110 over 8 weeks. 4 out of 10 R6/2 mice treated with TAT died whereas 0 out of 10 R6/2 mice treated with P110 died at 13 weeks old; no death occurred in 10 TAT- and 10 P110-treated WT mice. The results are shown as log-Rank (mantel-cox) test, chi square=9.461, p=0.0238).

FIG. 4: DNA damage measurement in urine and inflammation markers in plasma of WT and R6/2 mice. (A) WT and R6/2 mouse urine samples were collected at the age 13 weeks that were treated with control TAT or with P110 for 8 weeks. The levels of 8-OHdG were measured by ELISA. Creatinine levels in respective urine samples were determined for normalization. Increased levels of 8-OHdG in the urine of R6/2 mice were normalized by P110 treatment. The data are presented as mean±SEM. (n=9/group WT TAT and P110; n=6 R6/2 TAT; n=7 R6/2 P110). (B) TNFα and (C) IL-6 levels measured by ELISA in mice plasma of respective mice treated with TAT and P110 for 8 weeks. Plasma was collected at 13 weeks of age. (TNFαn=7/group WT TAT and P110; n=9 R6/2 TAT; n=10 R6/2 P110. IL-6: n=4/group WT TAT and P110; n=9 R6/2 TAT; n=10 R6/2 P110). The data are presented as mean±SEM.

FIG. 5: P110 reduces mtHtt aggregation in R6/2 skeletal muscle. Skeletal muscle sections were stained with anti-mtHtt (EM-48) antibody and hematoxylin (blue nuclei). Aggregates of mtHtt are shown (arrows) at a higher levels in TAT treated than P110 treated mice (20× magnitude). Bottom panels show magnification of boxed areas.

FIG. 6: P110 reduces mtHtt aggregation in R6/2 skeletal muscle. Skin sections were analyzed for the presence of mtHtt in WT and R6/2 mice. P110 reduced the level of mtHtt aggregates. Sections were viewed at 20× magnitude. Bottom panels show magnification of boxed areas.

FIG. 7: mtND2 levels in human HD CSF and plasma. mtND2 was determined in 3 CSF human samples from non-HD patients or from HD patients respectively. (A) Scatter plot illustrates the Cr values of nDNA GAPDH (X axis) against Cr values of mtDNA mtND2 (Y axis) in human CSF of non-HD and HD patients. R square value=0.9991 and 0.8277 for non-HD and HD subjects respectively. (B) mtND2 levels are shown in non-HD and HD patients CSF. The results between the 2 groups are not significant due to the small number of samples. (C) Distribution among the groups of human plasma mtND2 levels analyzed as above. Scatter plot illustrates Cr values of GAPDH (X axis) against Cr values of mtND2 (Y axis) of non-HD, pre-manifest and HD subjects. Cr values of mtND2 of HD plasma were lower and clustered below the 50% line compared to pre-manifest HD and non-HD groups. (D) mtND2 levels were significantly higher in HD plasma vs non-HD plasma (p=0.0415; n=6/non-HD and HD, n=5/pre-man). The data are presented as mean±SEM of 2^{-ΔΔC}T.

FIG. 8: (A) R6/2 mice have lower number of entries in the arms during the Y-maze spontaneous alternation test when compared to the WT mice. Their percentage of alternation is not statistically higher than 50% chance level while the WT mice have significantly higher alternation when compared to chance level. (B) During sociability phase of PhenoLab, both the WT and R6/2 mice spent significant amount of time in the zone with stranger 1 mouse when compared to the zone with a novel object indicating normal social behavior in both groups of mice. (WT novel object vs WT Stranger 1 p=0.0014, paired t-test; R6/2 novel object vs R6/2 Stranger 1 p=0.0001, paired t-test). However, the R6/2 mice were not spending significantly more time in the stranger 2 zone compare to stranger 1 zone indicating their lack of social discrimination to a novel stranger 2 mouse. WT mice spent significantly more time in stranger 2 zone compared to the stranger 1. (WT Stranger 1 vs WT Stranger 2 p=0.0117, nonparametric paired t-test; R6/2 Stranger 1 vs R6/2 Stranger 2, p=0.01852, paired t-test). (C) Both WT and R6/2 mice have comparable latency to enter the dark chamber during habituation and training day. WT mice have significantly longer latency to enter the dark chamber on Day 1 post training when compared to the R6/2 mice (p=0.0162, Mann-Whitney test). The difference in latency to enter the dark chamber was less prominent at Day 7 post training (p=0.0995, unpaired t-test). (D) The WT and R6/2 mice have similar percentage of freezing during Day 1 training and Day 2 cued testing. The percent freezing for the WT were significantly higher during Day 3 contextual testing when compared to the R6/2 mice (p=0.007, unpaired t-test).

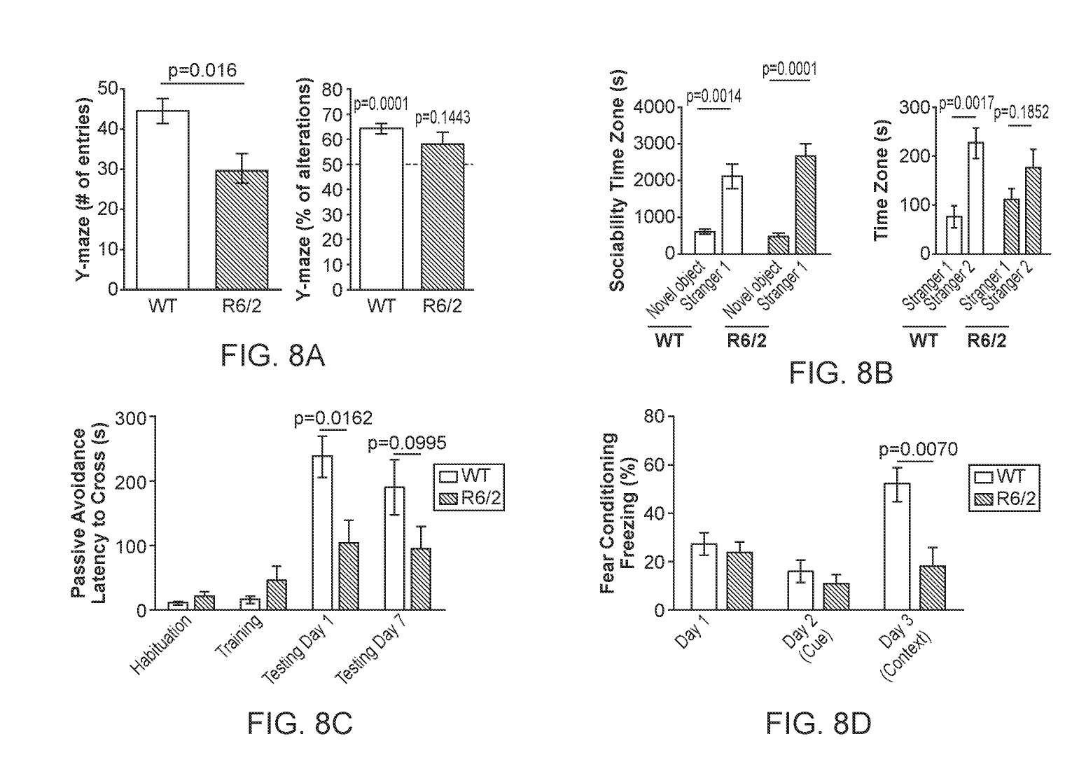

FIGS. 9A-9B: mtDNA levels in plasma of YAC128 mice. (A) Analysis of mtDNA levels (mtND2) in plasma of 6-mo-old untreated WT and untreated YAC128 mice. n=9 WT and n=9 YAC128. *, P=0.0115 WT versus YAC128. (B) Beneficial effect of 1-wk P110 treatment of 13-wk-old R6/2 mice. Circulating mtND2 levels were measured in plasma of R6/2 mice treated with P110 for 1 wk at 8 wk old before collection of the samples. The levels of mtND2 increased in P110-treated plasma compared with untreated. n=5 R6/2 TAT and n=3 P110 R6/2. The results are presented as mean±SEM of 2^{-Δ Δ CC}T. P=0.0146 TAT versus P110.

FIG. 10: 4-HNE staining of skeletal muscle and skin sections of 13 week old mice. (A) Protein adducts stained with 4-HNE is found predominantly in R6/2 muscle section (A) and skin section (B) relative to WT mice. Micrographs are shown at 20× magnification. Representative result of 6 sections of 4 mice/group.

### DETAILED DESCRIPTION

### Definitions

As used herein, the terms “treatment,” “treating,” and the like, refer to administering an agent, or carrying out a procedure for the purposes of obtaining an effect. “Treatment,” as used herein, covers any treatment in a mammal, particularly in a human, and includes: inhibiting ongoing neurodegenerative disease, i.e., arresting its development; and relieving neurodegenerative disease, i.e., causing regression.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction or elimination of the disease, symptoms of the disease, or side effects of the disease in the subject, and includes demonstration of effective changes in surrogate markers disclosed herein. A delay in the disease, or side effects of the disease, for example in an asymptomatic subject can also be monitored.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of side effects associated with standard or non-standard treatments of a disease. Determination of treatment efficacy is usually indication and disease specific and includes measuring the surrogate biomarkers described herein. Treatment efficacy may further be measured by assessing general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

The terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “nucleic acid molecule” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

“Substantially pure” indicates that an entity (e.g., a synthetic peptide or a mitochondrial fission inhibitor peptide or construct) makes up greater than about 50% of the total content of the composition (e.g., total protein of the composition), or greater than about 80% of the total protein content. For example, a “substantially pure” refers to compositions in which at least 80%, at least 85%, at least 90% or more of the total composition is the entity of interest (e.g. 95%, 98%, 99%, greater than 99%), of the total protein. The protein can make up greater than about 90%, or greater than about 95% of the total protein in the composition.

The terms “subject,” “individual,” and “patient” are used interchangeably herein to refer to a member or members of any mammalian or non-mammalian species that may have a need for the pharmaceutical methods, compositions and treatments described herein. Subjects and patients thus include, without limitation, primate (including humans and non-human primates), canine, feline, ungulate (e.g., equine, bovine, swine (e.g., pig)), avian, and other subjects. In some cases, the subject is a murine (e.g., rat or mouse) subject, such as a rat or mouse model of a disease. In some cases, the subject is a human.

The terms “mitochondrial fission inhibitor peptide,” “mitochondrial inhibitory peptide,” “mitochondrial inhibiting peptide,” are used interchangeably herein to refer to peptides previously described in, e.g. Qi et al. (2013) J. Cell Sci. 126(Pt 3):789-802; and US patent application US20130053321, each herein specifically incorporated by reference. As used herein, the term “therapeutic drug” or “therapeutic regimen” refers to an agent or protocol for administration of an agent, used in the treatment of a disease or condition, particularly a neurodegenerative condition for the purposes of the present invention. Of interest are clinical trials using such therapies, and monitoring of patients undergoing such therapy.

In some embodiments the therapeutic peptide comprises, consists or consists essentially of (i) YGRKKRRQRRR (SEQ ID NO:9), (ii) GG, and (iii) DLLPRGS (SEQ ID NO: 10) attached in order (i), (ii), and (iii) from amino terminus to carboxyl terminus.

A mitochondrial fission peptide, or genetic construct encoding a mitochondrial fission peptide, which may be monitored in a clinical trial format, will have one or more of the following activities: 1) inhibition of Drp1 GTPase activity; 2) inhibition of binding of Drp1 to Fis1; 3) reduction of mitochondrial damage in a cell under pathological conditions or other conditions of stress; 4) reduction of cell death in a cell under pathological conditions or other conditions of stress; 5) reduction of translocation of Drp1 from the cytosol to a mitochondrion; 6) and inhibition of mitochondrial fragmentation in a cell under pathological conditions. Other effects include, but are not limited to, reduced mitochondrial fragmentation in neuronal cells exposed to several mitochondrial toxins; reduced mitochondrial ROS(O₂—) production and subsequently improved mitochondrial membrane potential and mitochondrial integrity; increased cell viability through reduction in apoptosis and autophagic cell death; and reduced loss of neurites in primary dopaminergic neurons in a Parkinsonism cell culture model through reduction in mitochondrial fragmentation and mitochondrial ROS production. In a preferred embodiment, treatment with or exposure to a mitochondrial fission inhibitor construct or peptide will have minimal effects on mitochondrial fission and cell viability of cells which are in non-stressed conditions or in a non-disease state.

In some embodiments, the inhibitor activity is selective, with respect to effects of the peptide or construct on a particular protein. In other embodiments, the inhibitor activity is selective in reducing mitochondrial damage, reducing cell death, reducing translocation of Drp1 from the cytosol to a mitochondrion, and/or inhibiting mitochondrial fragmentation when used to treat a diseased or stressed cell as compared to when the same inhibitor peptide or construct is used to treat a healthy or non-stressed cell. For the purposes of the present disclosure, a diseased cell includes a healthy cell which has been treated or genetically engineered to model a diseased cell.

The term “patient sample” or “sample” as used herein refers to a sample from an animal, most preferably a human, seeking diagnosis or treatment of a disease, e.g. a neurodegenerative disease. Samples of the present invention include, without limitation, urine, saliva, breath, CSF, and blood, including derivatives of blood, e.g. plasma, serum, etc.; and peripheral tissue, e.g. skin, muscle, etc. In some embodiments a patient sample is a non-CNS peripheral sample. In some embodiments a patient sample is cerebrospinal fluid (CSF).

Sample Analysis.

Patient samples are analyzed to determine the levels of one or more analytes of interest as disclosed herein, e.g. mtDNA, markers of inflammation, markers of oxidative DNA damage, e.g. 8-OHdG; mtHtt aggregates; protein oxidation markers, for example 4-HNE adducts; etc. Methods of analysis include, without limitation, quantitative PCR, ELISA, immunohistochemistry, liquid chromatography-mass spectroscopy; HPLC; ion-monitoring gas chromatography/mass spectroscopy; gas chromatography; semiconductive gas sensors; immunoassays; mass spectrometers (including proton transfer reaction mass spectrometry), infrared (IR) or ultraviolet (UV) or visible or fluorescence spectrophotometers (i.e., non-dispersive infrared spectrometer); binding assays involving aptamers or engineered proteins etc. In some embodiments, the biological sample is patient urine or plasma.

### Methods

Conditions of interest for monitoring methods of the present invention include a variety of neurodegenerative conditions. In some embodiments of the invention, a patient is diagnosed as having a neurodegenerative condition, for which treatment is contemplated. The patient may be initially tested for activity prior to treatment, in order to establish a baseline level of activity. Alternatively, the patient may be released from a treatment regimen for a period of time sufficient to induce a neurodegenerative state, in which state the patient is tested for activity in order to establish a baseline level of activity.

To measure an increase or decrease of an activity or function upon treatment by a composition described herein, it is understood by the person having ordinary skill in the art that the function or activity can be measured, for example, in the presence and in the absence of the composition (e.g., mitochondrial fission inhibitor peptide, or construct) or before or after administration, and a comparison is made between the levels of the activities in the presence and absence of the composition. Alternatively, the function or activity can be measured, for example, in the presence of two separate compositions, and the levels of the activity or function in the presence of each composition are compared. An inhibition of an activity can be a reduction of about 5% to 10%, 5% to 20%, 2% to 20%, 10% to 20%, 5% to 25%, 20% to 50%, 40% to 60%, 50% to 75%, 60% to 80%, 75% to 95%, 80% to 100%, 50% to 100%, 90% to 100%, or 85% to 95% when comparing the two conditions. Similarly, activation of an activity can be an increase of about 5% to 10%, 5% to 20%, 2% to 20%, 10% to 20%, 5% to 25%, 20% to 50%, 40% to 60%, 50% to 75%, 60% to 80%, 75% to 95%, 80% to 100%, 50% to 100%, 90% to 100%, 85% to 95%, or more than 100% but less than 500%, when comparing the two conditions.

Depending on the subject and condition being treated and on the administration route, an active agent (e.g., a mitochondrial fission inhibitor peptide or construct) may be administered in dosages of, for example, 0.1 μg to 500 mg/kg body weight per day, e.g., from about 0.1 μg/kg body weight per day to about 1 μg/kg body weight per day, from about 1 μg/kg body weight per day to about 25 μg/kg body weight per day, from about 25 μg/kg body weight per day to about 50 μg/kg body weight per day, from about 50 μg/kg body weight per day to about 100 μg/kg body weight per day, from about 100 μg/kg body weight per day to about 500 μg/kg body weight per day, from about 500 μg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 250 mg/kg body weight per day, or from about 250 mg/kg body weight per day to about 500 mg/kg body weight per day. The range is broad, since in general the efficacy of a therapeutic effect for different mammals varies widely with doses generally being 20, 30 or even 40 times smaller (per unit body weight) in man than in the rat. Similarly the mode of administration can have an effect on dosage. Thus, for example, oral dosages may be about ten times the injection dose. Higher doses may be used for localized routes of delivery. In some embodiments a peptide inhibitor is delivered by injection.

A specific mitochondrial fission inhibitor peptide or construct can be administered in an amount of from about 1 mg to about 1000 mg per dose, e.g., from about 1 mg to about 5 mg, from about 5 mg to about 10 mg, from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

The mitochondrial fission inhibitor peptide or construct can be administered with an intermittent dosing regimen, whereby treatment periods are interrupted by rest periods wherein the mitochondrial fission inhibitor peptide or construct is not administered. Non-limiting examples of intermittent dosing may include, for example, about one week of treatment, about two weeks of treatment, about one month of treatment, about two months of treatment, and the like, followed by about one week of non-treatment, about two weeks of non-treatment; about three weeks of non-treatment, about one month of non-treatment, about two months of non-treatment, about three months of non-treatment, and the like. In one embodiment, about one week of treatment is followed by three weeks of non-treatment. In another embodiment, one month of treatment is followed by 3 months of non-treatment. The rest periods may permit, for example, recovery from side effects due to administration of the therapeutic agent, reduced use of the therapeutic agent; reduced cost of treatment, etc.

The ability of an individual to respond to a candidate therapy for a neurodegenerative disease, e.g. HD, is analyzed by obtaining a pre-treatment sample; administering the candidate therapy; and obtaining one or more post-treatment sample. The level of one or more peripheral biomarkers described herein is determined, and the change in the patient sample is determined.

Patient samples include a variety of bodily fluids, e.g. blood and derivatives thereof, urine, saliva, breath, etc. The samples will be taken prior to treatment, and at suitable time points following administration, e.g. at 1 day, 2 days, 3 days, 1 week, 2 weeks, 3 weeks, or more, following administration.

In some preferred embodiments, the methods of the invention are used in determining the efficacy of a therapy for treatment of a neurodegenerative disease, either at an individual level, or in the analysis of a group of patients, e.g. in a clinical trial format. Such embodiments typically involve the comparison of two time points for a patient or group of patients. The patient status is expected to differ between the two time points as the result of a therapeutic agent, therapeutic regimen, or disease challenge to a patient undergoing treatment.

Examples of formats for such embodiments may include, without limitation, testing for the level of biomarkers at two or more time points, where a first time point is a diagnosed but untreated patient; and a second or additional time point(s) is a patient treated with a candidate therapeutic agent or regimen.

In another format, a first time point is a diagnosed patient in disease remission, e.g. as ascertained by current clinical criteria, as a result of a candidate therapeutic agent or regimen. A second or additional time point(s) is a patient treated with a different candidate therapeutic agent or regimen or with placebo.

In such clinical trial formats, each set of time points may correspond to a single patient, to a patient group, e.g. a cohort group, or to a mixture of individual and group data. Additional control data may also be included in such clinical trial formats, e.g. a placebo group, a disease-free group, and the like, as are known in the art. Formats of interest include crossover studies, randomized, double-blind, placebo-controlled, parallel group trial is also capable of testing drug efficacy, and the like. See, for example, Clinical Trials: A Methodologic Perspective Second Edition, S. Piantadosi, Wiley-Interscience; 2005, ISBN-13: 978-0471727811; and Design and Analysis of Clinical Trials: Concepts and Methodologies, S. Chow and J. Liu, Wiley-Interscience; 2003; ISBN-13: 978-0471249856, each herein specifically incorporated by reference.

In one embodiment, a blinded crossover clinical trial format is utilized. A patient alternates for a set period of time, e.g. one week, two weeks, three weeks, or from around about 7-14 days, or around about 10 days, between a test drug and placebo or a test agent and a different therapeutic agent, with a 4-8 week washout period.

In another embodiment a randomized, double-blind, placebo-controlled, parallel group trial is used to test drug efficacy. In one embodiment, individuals identified as having HD genotype undergo sequential treatment periods, each of 1-14 day durations. Subjects will be assessed at entry and at the end of each treatment period. During the first treatment period (run-in), all subjects will receive placebo. During the second treatment period, the subjects will be randomized into drug or placebo groups. During the third treatment period, subjects will remain on the same (drug or placebo) treatment as in the second period. Drugs that are effective will show a statistically lower frequency of relapse in the treatment arm versus placebo arm of the study.

Measurement of nucleic acids in peripheral blood, e.g. mitochondrial DNA, or mitochondrial mRNA in plasma may utilize any suitable mitochondrial sequence. The human mitochondrial genome has been sequenced, see Anderson et al. (1981) Nature 290, 457-465, and provides suitable primers for sequence detection. In some embodiments the sequence identification is drawn to one of the polypeptide coding sequences in mtDNA, for example sequences encoding NADH dehydrogenase; ATP synthase; cytochrome c oxidase; ubiquinol cytochrome c reductase, etc. The complete mitochondrial genome sequence can be accessed at Genbank, locus HUMMTCG, accession number J01415. Coding sequences include NADH dehydrogenase subunit 1, residues 3307-4262; NADH dehydrogenase subunit 2 at residues 4470-5511; cytochrome oxidase subunit 1 at residues 5904-7445; cytochrome oxidase subunit 2 at residues 7586-8269; ATPase8 at residues 8366-8572; ATPase6 at residues 8527-9207; cytochrome oxidase subunit 3 at residues 9207-9990; NADH dehydrogenase subunit 3 at residues 10059-10404; NADH dehydrogenase subunit 4 L at residues 10470-10766; NADH dehydrogenase subunit 4 at residues 10760-12137; NADH dehydrogenase subunit 5 at residue 12337-14148; NADH dehydrogenase subunit 6 at residues 14149-14673; and cytochrome b at residues 14747-15887.

A hematologic sample, e.g. a sample comprising blood cells, is obtained from a subject. Examples of hematologic samples include, without limitation, a peripheral blood sample and derivatives thereof, e.g. plasma, serum, and the like. A sample that is collected may be freshly assayed or it may be stored and assayed at a later time. If the latter, the sample may be stored by any means known in the art to be appropriate in view of the method chosen for assaying mtDNA. For example the sample may freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. for a blood sample, to purify plasma or serum. Further fractionation may also be performed, e.g., for a plasma or serum sample, fractionation based upon size, charge, mass, or other physical characteristic may be performed to purify particular secreted nucleic acids.

Exemplary methods known in the art for measuring mRNA or DNA levels in a sample include hybridization-based methods, e.g. southern blotting, northern blotting and in situ hybridization (Parker & Barnes, Methods in Molecular Biology 106:247-283 (1999)), RNAse protection assays (Hod, Biotechniques 13:852-854 (1992)), RNAseq, PCR-based methods (e.g. quantitative PCR (q-PCR).

For measuring mtRNA or mtDNA levels, general methods for nucleic acid extraction are well known in the art and are disclosed in standard textbooks of molecular biology, including Ausubel et al., Current Protocols of Molecular Biology, John Wiley and Sons (1997). Isolation can also be performed using a purification kit, buffer set and protease from commercial manufacturers, according to the manufacturer's instructions. For example, RNA from cell suspensions can be isolated using Qiagen RNeasy mini-columns, and RNA or DNA can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.), RNA Stat-60 kit (Tel-Test), etc.

Hybridization assays in which a nucleic acid that displays “probe” nucleic acids for each of the sequences to be assayed/profiled in the profile to be generated may be employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

For measuring proteins, e.g. 4-HNE adducts or markers of DNA damage, e.g. 8-OHdG, the amount or level of one or more such analytes in the sample is determined. In such cases, any convenient protocol for evaluating analyte levels may be employed.

While a variety of different manners of assaying for analyte levels are known in the art, one representative and convenient type of protocol is ELISA. In ELISA and ELISA-based assays, one or more antibodies specific for the analyte of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 250-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more analytes in a sample may be employed. Representative examples include but are not limited to mass spectrometry, proteomic arrays, xMAP™ microsphere technology, western blotting, immunohistochemistry, and flow cytometry. In, for example, flow cytometry methods, the quantitative level of analytes are detected in cells in a cell suspension by lasers. As with ELISAs and immunohistochemistry, antibodies (e.g., monoclonal antibodies) that specifically bind the analyte are used in such methods.

The methods of the invention also find use in preclinical analysis. Suitable non-human animal models of Parkinson's disease (PD) include, e.g., the α-synuclein transgenic mouse model; and the 1-methyl-4-phenyl-1,2,3,6,-tetrahydropyridine (MPTP) mouse model of Parkinson's disease. See, e.g., Betarbet et al. (2002) Bioessays 24:308; Orth and Tabrizi (2003) Mov. Disord. 18:729; Beal (2001) Nat. Rev. Neurosci. 2:325.

Suitable non-human animal models of Huntington's disease include, e.g., a transgenic mouse comprising a human huntingtin transgene (e.g., the R6 line, the YAC line), where the human huntingtin transgene comprises 30-150 CAG repeats (encoding a polyglutamine expansion); a knock-in mouse model, comprising a homozygous or heterozygous replacement of endogenous mouse huntingtin gene with a human huntingtin gene comprising 30-150 CAG repeats. See, e.g., Mangiarini et al. (1996) Cell 87:493; Menalled (2005) NeuroRx 2:465; and Menalled and Chesselet (2002) Trends Pharmacol. Sci. 23:32; and Hodgson et al. (1999) Neuron 23:181.

In addition to surrogate biomarkers, the effect of a candidate therapy on cognitive function, muscle function, motor function, brain function, behavior, and the like, can be assessed. Electrophysiological tests can be used to assess brain function. Muscle function can be assessed using, e.g., a grip strength test. Motor function can be tested in rodents using, e.g., a rotarod test. Cognitive functions can be tested for rodents using, e.g., the open field test, the elevated plus maze, the Morris water maze, the zero maze test, the novel objection recognition test, and the like. Tests for neurological functioning and behavior that include sensory and motor function, autonomic reflexes, emotional responses, and rudimentary cognition, can be carried out. Such tests are well known in the art; see, e.g., Chapter 12 “Assessments of Cognitive Deficits in Mutant Mice” by Rodriguiz and Wetsel, in “Animal Models of Cognitive Impairment” (2006) E. D. Levin and J. J. Buccafusco, eds. CRC Press, Boca Raton, Fla.

Primary outcome measures used in clinical trials of patients with diagnosed HD typically involve a clinical symptom or sign (e.g., chorea) and a measure of everyday function. One of the most frequently used measures of function in HD is the Total Functional Capacity scale (TFC). (see Shoulson and Fahn (1979) Neurology 29(1):1-3. The Symbol Digit Modalities Test (SDMT) measures the number of correct responses on a timed task of symbol to digit transcription and taps psychomotor speed, attention, and working memory. Higher scores indicate better cognitive functioning (see Smith A. Symbol Digit Modalities Test Manual. Western Psychological Services; Los Angeles, Calif.: 1973). Motor signs (e.g., finger tapping, chorea, dysarthria) can be assessed the UHDRS (Unified Huntington's Disease Rating Scale: reliability and consistency. Huntington Study Group. Mov Disord. 1996; 11(2):136-142). Other tests useful in the evaluation of HD may include, for example, the Zung Depression Sale; Mini Mental State Examination (MMSE); the Barthel Index; the Tinetti performance Oriented Mobility Assessment (POMA); the Thurstone Word Fluency Test (TWFT); the Stroop test, etc.

### Databases of Analyses

Also provided are databases of analyses of peripheral biomarkers. Such databases will typically comprise analysis profiles of various individuals following a clinical protocol of interest etc., where such profiles are further described below.

The profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression profile.

Also provided are reagents and kits thereof for practicing one or more of the above-described methods. The subject reagents and kits thereof may vary greatly. Reagents of interest include reagents specifically designed for use in production of the above described analysis. Kits may include reagents for analysis of biological sample, e.g. primers for PCR amplification, antibodies for detection of proteins and adducts, and such containers as are required for sample collection.

The kits may further include a software package for statistical analysis. In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The treatment response patterns from individuals or groups of individuals can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature, and the like), but some experimental errors and deviations may be present. Unless indicated otherwise, parts are parts by weight, molecular weight is weight average molecular weight, temperature is in degrees Centigrade, and pressure is at or near atmospheric.

### Example 1

### Biomarkers to Follow the Progression and Treatment Response of Huntington's Disease

Reference may be made to Disatnik et al. (2016) J. Exp. Med. 213(12):2655-2669, entitled “Potential biomarkers to follow the progression and treatment response of Huntington's disease”, herein specifically incorporated by reference in its entirety.

We recently reported that inhibition of mitochondrial dynamics impairment by a novel Drp1/Fis1 peptide inhibitor, P110, rescued mtHtt-induced mitochondrial injury, corrected defects in mitochondrial function, and reduced neuronal cell death both in HD patient-derived neuronal cultures and in HD transgenic mouse brains. These findings provided further evidence for a causal role for mitochondrial damage in the pathogenesis of HD, and demonstrated that blocking mitochondrial injury can reduce neuronal degeneration in HD models. Here we used samples from R6/2 mice as a HD model to identify biomarkers that correlate with HD disease progression and treatment benefit with P110, providing a reasonable model to predict therapeutic efficacy. We also include a pilot human study for one of these biomarkers, using plasma and spinal fluid samples from healthy subjects and HD patients.

Alteration of mtDNA in the Brain and Plasma of HD Mice.

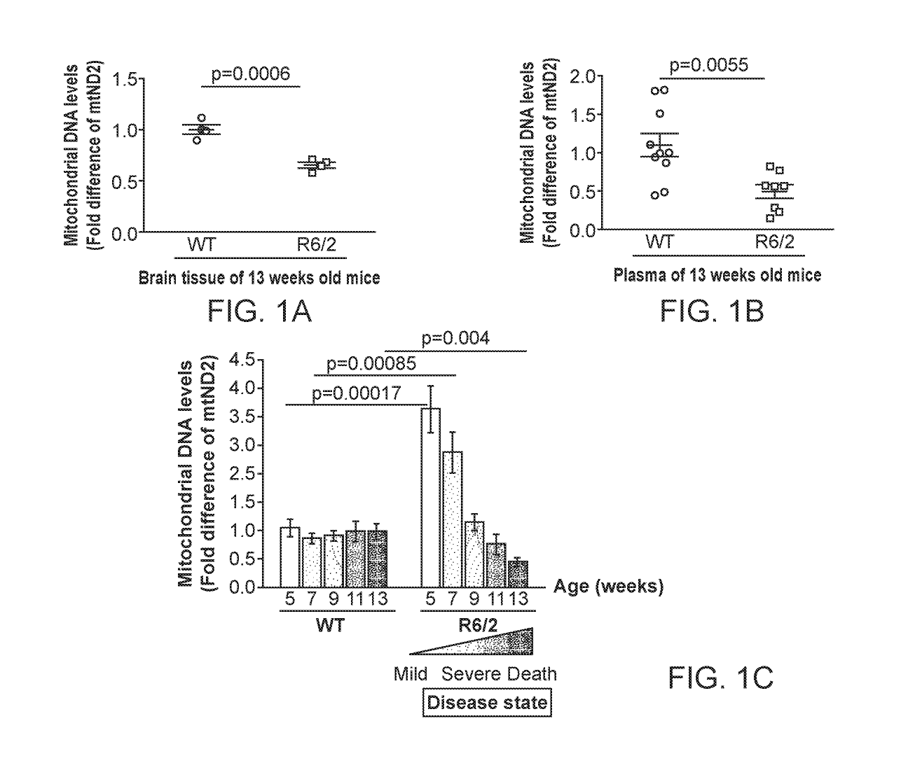

Since Huntington's disease is associated with impaired mitochondrial integrity and excessive mitochondrial fission, we first evaluated the extent of mitochondrial loss in the brain of 13-week old R6/2 mice, an age that we previously found to exhibit severe HD-related symptoms. It was previously found that mitochondrial number in the brain decline by more than 50% in severe HD patients. As a surrogate measure for mitochondrial number in the brain, we measured the levels of the transcript of the mitochondrial gene, mtND2 (mitochondria encoded NADH dehydrogenase 2; a subunit of complex 1 located at the inner mitochondrial membrane), using DNA reverse transcribed from RNA isolated from brain tissue of wild type (WT) and R6/2 mice. Using real-time PCR, we found that the brain of 13 weeks old R6/2 mice had almost half the amount of mtND2 as compared with brains of WT mice (FIG. 1A).

Since mitochondrial DNA is quite resistant to degradation, we expected that the content of mitochondrial DNA in the plasma to increase as dead neurons release mitochondrial DNA into the circulation. We therefore determined the levels of mitochondrial DNA in the plasma of 13 weeks old mice, using the mtND2 transcript as above. GAPDH, a nuclear gene, was used as a control. Surprisingly, mtND2 levels in R6/2 plasma were reduced by 54% as compared with plasma of WT mice (FIG. 1B).

A candidate biomarker should show a measurable response to the progression and severity of the disease. Thus, to evaluate the levels of mtND2 in mouse plasma during the course of 13 weeks, we collected plasma every 2 weeks and analyzed by real-time PCR mtND2 levels to determine whether they correlate with the progression of the disease. The results are shown as the averages of mtND2 levels (2^{-ΔΔC}T) obtained from 10 mice per group at each age (FIG. 1C). We observed that mtND2 levels in plasma of WT mice remained constant over time from 5 to 13 weeks of age. However, mtND2 levels in R6/2 mice were 3.5-fold higher at 5 weeks and 2.5-fold higher at 7 weeks as compared with WT mice and these levels decreased over time, to half of the WT levels, by 13 weeks (FIG. 1C).

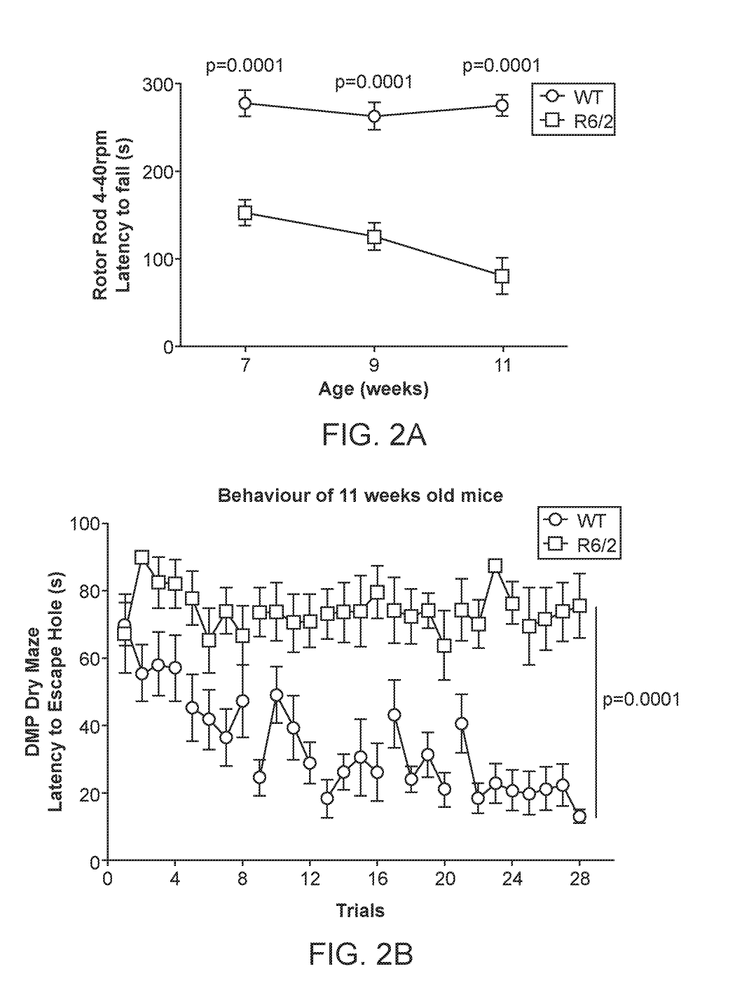

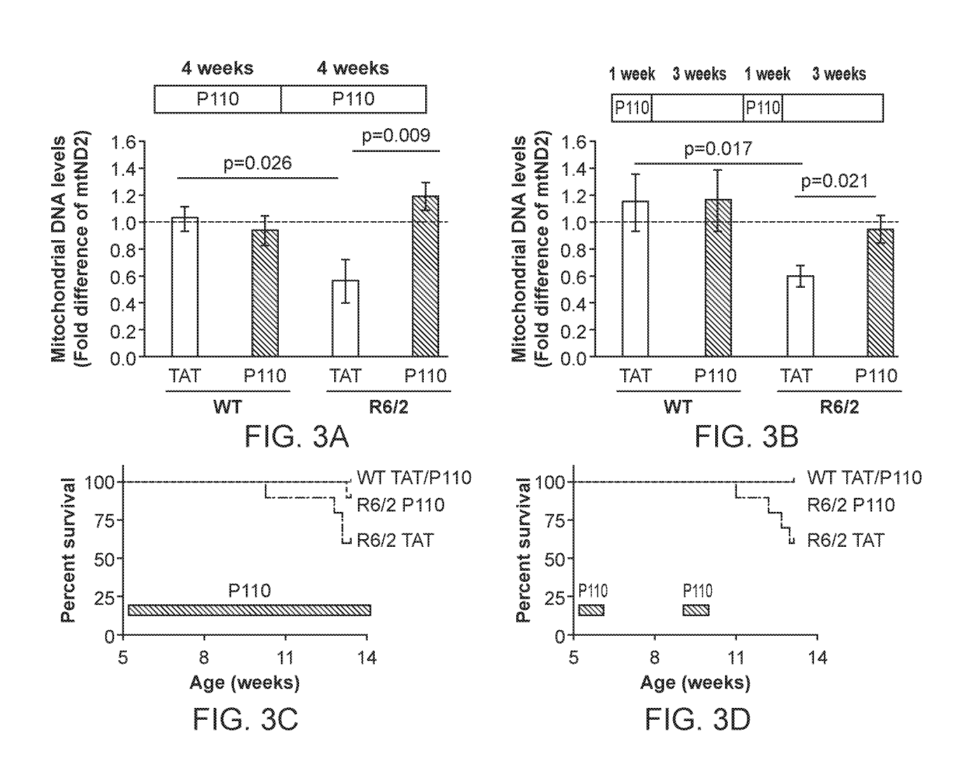

We previously reported that 13 weeks old R6/2 HD mice exhibited a severe behavioral deficit accompanied by mitochondrial loss (FIG. 1B). Behavior studies measured by several tests of 7 weeks old mice demonstrated an overall behavioral deficit of R6/2 mice as compared with WT mice (FIGS. 2A and B and FIG. 3 A-D). The behavioral deficit, shown by a decrease in mobility (FIG. 2A) measured at 7 weeks of age, which became more severe with age. [Note that younger behavioral studies in mice that have not been acclimated to animal facility were less reliable. However, on arrival, at age of 5 weeks, the HD mice appear relatively unimpaired]. 11 week old R6/2 mice demonstrated high deficiency of memory and learning skills as shown by Delay Match-to-Place (DMP) dry maze (FIG. 2B). No behavioral deficits were noted in 5 week old R6/2 mice. Yet, 5 week old R6/2 mice have high levels of mtDNA in plasma relative to WT controls (FIG. 1C). These results suggest that evidence for maximal neuronal loss (as measured by decreased mitochondria DNA in the brain and increased mitochondrial DNA in the plasma) occurred earlier than motor and behavioral impairments. Taken together, these results show that the highest increase in mitochondrial DNA in the plasma preceded maximal behavioral deficits of R6/2 mice, consistent with the evidence that mitochondrial damage occurred at an early stage of HD. Therefore, assessing mtDNA in the plasma may be a useful marker to indicate early HD-associated pathology.

P110 Treatment Normalizes the a Mount of mtDNA in the Plasma of HD Mice.

We previously described the beneficial effect of P110 treatment on HD mice. P110 is a heptapeptide conjugated to TA₄₇₋₅₇ that inhibits the interaction between Drp1 and one of its adaptor proteins in the mitochondria, Fis1. We showed that P110 inhibits excessive mitochondrial fission in several models of neurodegeneration disease as well as in a rat heart model of ischemia/reperfusion injury, without affecting basal (physiological) fission. To determine whether mitochondrial DNA levels in the plasma correlated with the benefit induced by P110, R6/2 mice were treated with P110 inhibitor peptide or TAT (vehicle control, each at 3 mg/Kg/day), delivered by a subcutaneous osmotic pump for 8 weeks, as we described before (Guo et al. (2013) J Clin Invest 123, 5371-5388). As in the cohort shown in FIG. 1, 13 week old R6/2 mice exhibited a decrease of 50% in mtND2 in the plasma and P110 treatment increased the levels of mtND2 levels by two folds, back to those of WT levels (FIG. 3A).

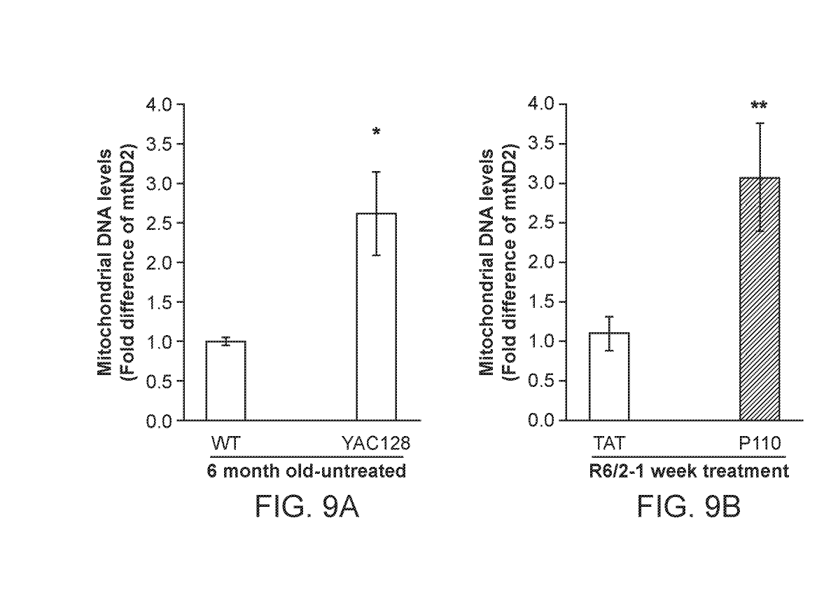

We next determined the effect of intermittent P110 treatment consisting of 1 week sustained treatment with P110 (3 mg/Kg/day) followed by no treatment for 3 weeks, repeated twice for a total duration of 8 weeks, as shown in FIG. 3B. We found that an intermittent P110 treatment was sufficient to increase the levels of mtND2 by two folds, close to wild type levels (FIG. 3B). As previously reported, we found that P110 administered for 8 weeks was beneficial and increased the survival of the R6/2 mice (FIG. 3C). We also found that survival of R6/2 mice subjected to an intermittent P110 treatment was also significantly increased (p=0.0238, FIG. 3D), indicating that intermittent treatment might be sufficient to correct mitochondrial function, thus protecting from neuron cell loss. Finally, we observed that P110 treatment for only one week in 8 weeks old R6/2 mice was sufficient to increase the levels of mtND2 in the plasma by two folds relative to TAT-treated R6/2 mice (FIG. 9A-9B), suggesting that this measure correlates with treatment.

P110 Treatment Reduces the Levels of Oxidative DNA Damage Indicator in HD Mice.

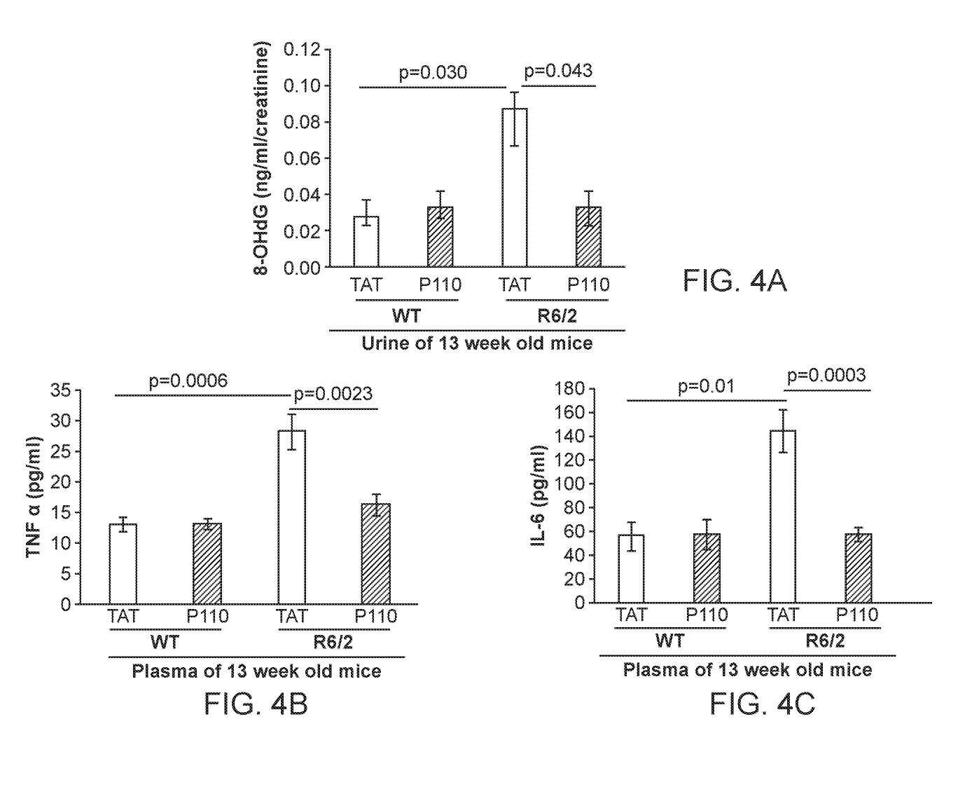

There are conflicting reports regarding the use of oxidative stress markers in plasma and urine, such as 8-hydroxy-deoxy-guanosine (8-OHdG). 8-OHdG is a product of guanine oxidation by oxidative stress that is found in the urine as a product of DNA excision repair. Urine and plasma from R6/2 mice have high levels of 8-OHdG. We therefore evaluated the use of 8-OHdG as a biomarker for treatment benefit in urine of WT and R6/2 mice after 8 weeks of P110- or TAT vehicle-treatments. (Note that because the mice are fragile, continual collection of urine as the disease progresses was not possible). The DNA damage product, 8-OHdG, measured by ELISA assay, was normalized to the levels of creatinine in each mouse urine sample, to accommodate differences in water intake and urine volume. We found that 8-OHdG levels were 3 fold higher in 13 weeks old R6/2 mice relative to WT mice of the same age and that an 8-week P110 treatment of the R6/2 mice decreased the levels of 8-OHdG to wild type levels (FIG. 4A).

P110 Treatment Decreases the Levels of Inflammatory Markers in Plasma of Hd Mice.

Activated monocytes are observed in the pre-symptomatic HD patients and inflammation triggered by the presence of mtHtt was reported in mouse models of HD and in HD patients. Inflammation is due, in part, to activation of microglia and recruitment of astrocytes associated with mtHtt, which leads to enhanced secretion of cytokines and chemokines by microglia. Therefore, using ELISA, we measured the levels of two inflammatory cytokines: TNFα and IL-6. The levels of both these cytokines were elevated in the plasma of 13 weeks old R6/2 mice by more than two folds relative to WT mice, and P110-treatment of R6/2 mice for 8 weeks reduced their levels back to the levels of WT mice (FIG. 4, B and C).

P110 Treatment Reduces the Levels of mtH tt Aggregation and 4-HNE Adducts in Peripheral Tissues of HD Mice.

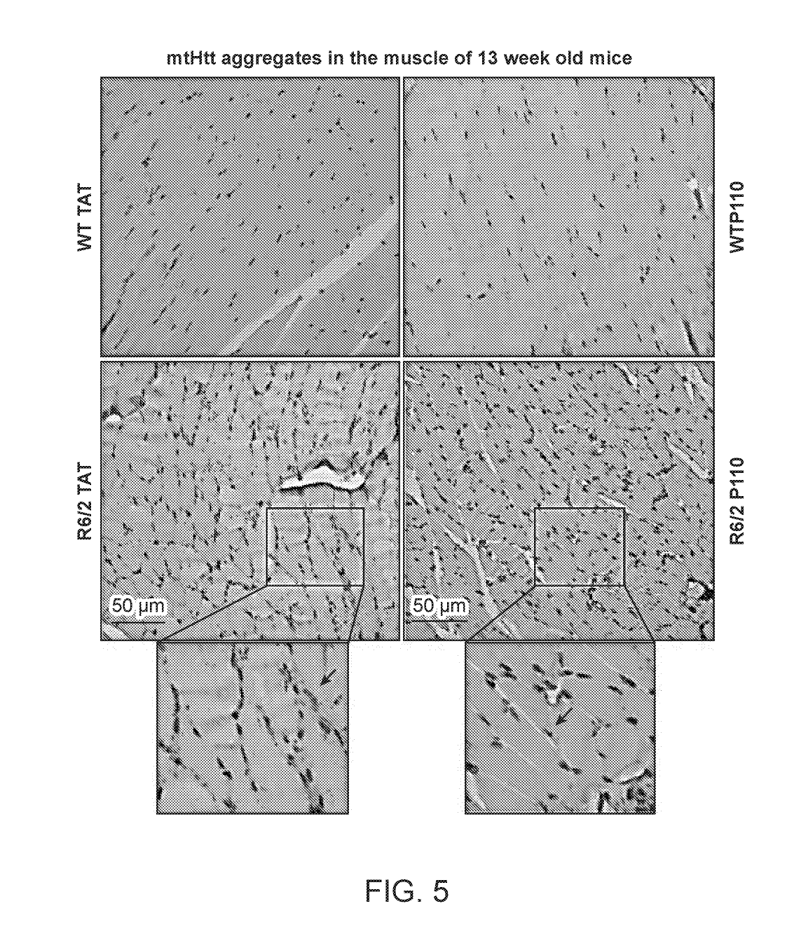

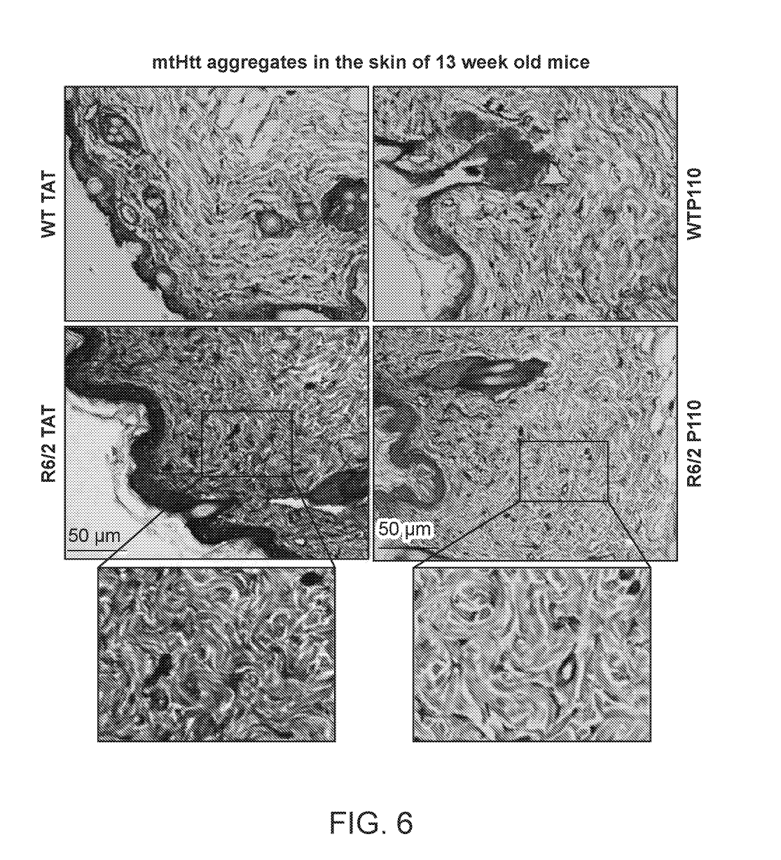

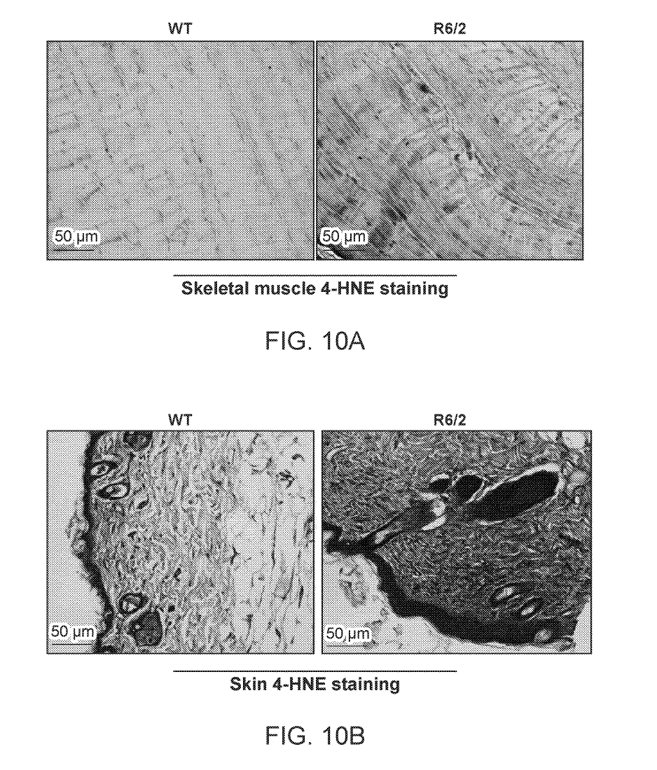

Aggregates of mtHtt were previously reported in the brain of human HD patients and R6/2 mice when measured at the age of 13 weeks. However, non-CNS tissues of HD mice model also have mtHtt aggregates as well as evidence of oxidative stress. We therefore determined the presence of mtHtt aggregates and 4-HNE adducts on proteins (an aldehydic product of lipid oxidation) in skeletal muscle and skin of 13 weeks old R6/2 mice. We found an increase in mtHtt aggregates at the periphery of the muscle fibers (FIG. 5) and skin sections (FIG. 6) in TAT-treated R6/2 mice; an 8-wk P110 treatment of R6/2 mice correlated with decreased levels of these aggregates by 40% in the skin and 60% in the muscle tissue (Table 1). We also found skeletal muscle from R6/2 compared with WT mice to have a high number of nonmuscle nuclei (FIG. 5) that might reflect infiltration of inflammatory cells into this tissue. We also observed twofold-higher levels of 4-HNE immunoreactivity in R6/2 leg muscle as well as 36% increase in skin sections stained with 4-HNE, as compared with WT levels (FIG. 10, A and B and Table 1). Those results present evidence that non-CNS peripheral tissue can be used to follow the progression of HD.

TABLE1
Biomakers in peripheral tissues

| | mtHtt staining | 4-HNE staining |
|---|---|---|
| Muscle | | |
| WT TAT | 80.8 ± 2.0 | 45.1 ± 2.8 |
| WT P110 | 94.8 ± 0.5 | |
| R6/2 TAT | 143.3 ± 0.4^{a} | 91.8 ± 2.5^{b} |
| R6/2 P110 | 57.1 ± 0.6^{c} | |
| Skin | | |
| WT TAT | 96.4 ± 4.2 | 95.3 ± 2.8 |
| WT P110 | 89.4 ± 3.3 | |
| R6/2 TAT | 141 ± 5.0^{d} | 148.6 ± 7.0^{e} |
| R6/2 P110 | 85.7 ± 3.5^{f} | |

Quantification of images of mtHtt and 4-HNE staining in muscle and skin. Images of the respective staining were obtained from three mice/group. and 16 areas of each section were analyzed. 58 areas of each section were analyzed for mtHtt staining in muscle. Data are presented as mean ± SEM.
^{a}P = 0.038 (WT TAT vs. R6/2 TAT)
^{b}P = 0.0026 (WT TAT vs. R6/2 TAT)
^{c}P = 0.027 (R6/2 TAT vs. R6/2 P110)
^{d}P = 0.019 (WT TAT vs. R6/2 TAT)
^{e}P = 0.018 (WT TAT vs. R6/2 TAT)
^{f}P = 0.015 (R6/2 TAT vs. R6/2 P110)

Alteration of mtDNA Content in Biofluids of HD Patients.

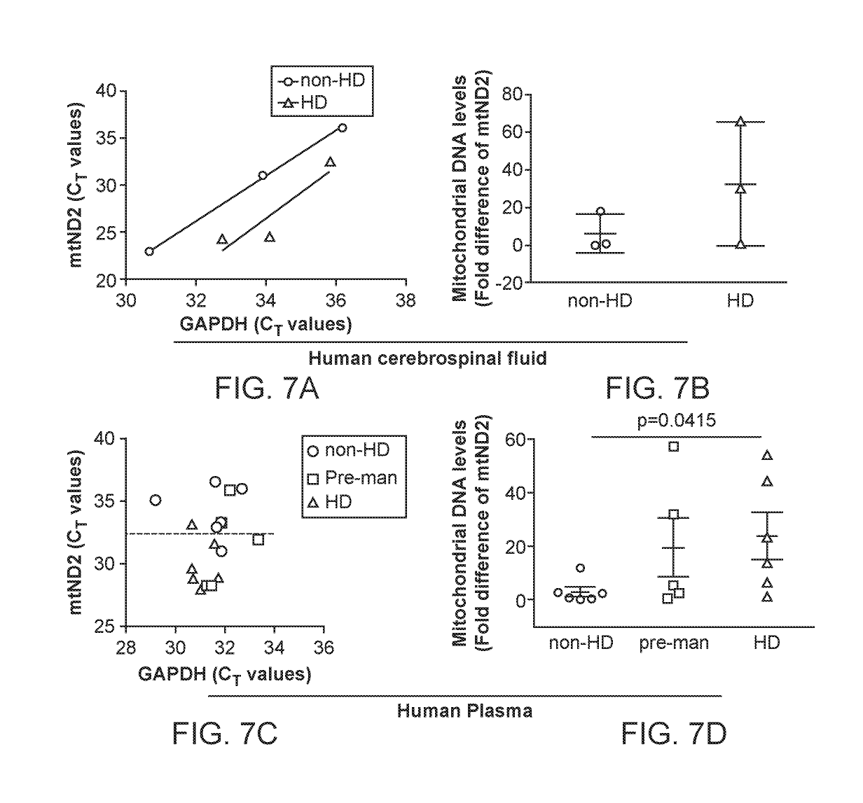

To begin determining whether the biomarkers identified in R6/2 mice can be applied in human studies, we obtained three each of human cerebrospinal fluid (CSF) samples collected from control or HD patients, ages 53 to 69 years old; both males and females were included (VA Greater Los Angeles Healthcare Center). Two of the control subjects were reported to have a chronic obstructive pulmonary disease. These CSF samples, the only samples that were available to us, were used in a pilot study; as described above, using real-time PCR, we measured the level of mtND2 levels in these samples. The differences in mtND2 levels were not significantly different between control and HD patients, probably because of the low number of samples, the wide range of age of the subjects and variations in disease onset. However, there is a shift in the correlation line to the right for the three HD patient samples (FIG. 7A). There was also a wider range of mtDNA levels in HD CSF samples as compared with non-HD patients (FIG. 7B).

We then determined the levels of mtDNA in the plasma of HD patients and control subjects. Again, we obtained only a few samples for the study (courtesy of Dr. Leavitt, University of British Columbia). However, even in this small sample group, we found a correlation between the severity of the disease and the levels of mitochondrial DNA (mtND2) measured in plasma of HD patients (FIG. 7, C and D); the increase shown in mtND2 in pre-symptomatic and symptomatic HD patients compared to control subjects was significant (p=0.0415) and may correlate with the results obtained in R6/2 mice at the ages of 5-7 weeks.

The challenge in conducting clinical trials using experimental therapeutics for HD patients is not the diagnosis of these patients—HD patients can unequivocally be identified via genetic testing for this dominant trait. The challenge is how a response to the experimental treatment can be assessed, considering that the main affected tissue responsible for the pathology is the brain. Furthermore, changes in affected individuals must occur from the time of conception, yet neurodegeneration symptoms are not apparent for more than 40 or 50 years. Therefore, although ideally, therapeutic interventions should begin in pre-symptomatic subjects, it is prohibitively expensive to await several decades to assess the benefit of that intervention.

Here we began exploring the possibility that peripheral biomarkers to assist in clinical trials in HD patients can be identified. We focused on biomarkers related to (i) mitochondrial and cell integrity (measuring mitochondrial DNA in the plasma), (ii) mtHtt aggregation in the peripheral tissue, and (iii) evidence of increased oxidative stress, as measured by increased aldehydic load in human and mice brain tissue [levels of 4-HNE adducts and DNA damage (as measured by the presence of a product of DNA repair in the urine—8-OHdG]. We found that the levels of all these parameters differed between WT mice and R6/2 HD mice. Importantly, the levels of these parameters in R6/2 mice were normalized by treatment with P110, a therapeutic intervention that reduces the symptoms and pathology in these animals; levels of these parameters in treated HD mice were brought close to WT levels. Therefore, all of these biomarkers appear to correlate with the improvement seen by the therapeutic intervention in this animal model.

When focusing on the presence of mtDNA in plasma, we also noted that both sustained and intermittent treatment (1 week on 3 weeks off, twice) were beneficial (FIG. 3, A and B) and a small study of one-week treatment at the onset of the disease (FIG. 9A-9B) also suggests that mitochondrial DNA in the plasma of these HD mice may correlate even with a short therapeutic intervention. mtDNA is a useful biomarker to assist in determining the efficacy of a treatment in humans.

Mitochondrial dysfunction in HD is well documented as a main contributor to neurodegeneration and is associated with the accumulation of mtHtt protein at the mitochondria and in the nucleus. Progressive loss of striatal and cortical neurons mediates the cognitive and motor impairments in HD patients and in R6/2 mice. Studies in R6/2 mice showed a decrease of brain weight at 4 weeks, thus preceding body weight loss and motor deficits. Reports in both HD patients and HD transgenic mice also revealed that deficits in energy metabolism attributable to mitochondrial toxin-induced mitochondrial dysfunction, play a key role in HD pathogenesis. Moreover, clinical evidence shows that metabolic impairment precedes neuropathology and clinical symptoms in HD patients, indicating that metabolic deficit is an early event in HD. Together our findings demonstrated that mitochondrial dysfunction and damage are associated with HD pathology.

Quantification of nuclear and mitochondrial DNA raised a great interest as a non-invasive diagnostic for patients after trauma and for diseases such as cancer and neurodegenerative diseases. Our study in plasma and CSF of HD patients shows statistically significant higher mtDNA plasma levels in plasma of HD patients as compared with control subjects (FIG. 7, A-D). Since clinical information on these patients is not available to us, the correlation with changes in this biomarker and the severity of the disease (as we found in R6/2 mice; FIG. 1) cannot be made.

Why do the plasma levels of mtDNA increase in HD mice and of patients with HD? The presence of mitochondrial DNA in the plasma may reflect lysis of neuronal cells (or other cells) and the release of their content into the plasma. mtDNA may reflect active exosome-mediated release of damaged mitochondria. The fact however that the levels of mtDNA rise and then decline over the course of the disease (FIG. 1C) probably reflect initially cell damage, and subsequently decline in cell and/or mitochondrial number. Future studies may address directly this question. Nevertheless, the finding that a mouse model of HD shows a dynamic change in this biomarker that responds to P110 treatment and correlates with therapeutic effect and the finding that the same biomarker is abnormal in humans with HD is encouraging.

As discussed above, the damage to mitochondria shown in HD patients and HD mice models causes oxidative damage to the DNA and can be measured by the presence of 8-OHdG, a principle marker of hydroxyl radical damage to DNA, in urine and plasma. However, there are conflicting reports on the use of 8-OHdG as an oxidative stress marker: Urine and plasma from R6/2 mice were found to have high levels of 8-OHdG, using HPLC with electrochemical detection analytic technique in the study. Borowsky et al. reported that 8-OHdG was not found to be a biomarker of disease progression when using either liquid chromatography-mass spectrometry (LCMS) assay or liquid chromatography-electrochemical array (LCECA) assay. Our data using ELISA method are consistent with the Bogdanov reports, showing a 2.5-fold increase in 8-OHdG in the urine of 13 weeks old R6/2 mice relative to age-matched WT mice and these were lowered to WT mouse levels in R6/2 mice treated with P110 for 8 weeks (FIG. 4A). Therefore, the presence of 8-OHdG in the urine is a biomarker for a trial of therapeutic intervention.

DNA oxidation and mitochondria release into circulating fluids provoke inflammation in HD. In addition, excessive mitochondrial fragmentation in microglial cells lead to a pro-inflammatory state in the brain vasculature, and in activated microglial cells and several HD mouse models as well as HD patients have increased plasma cytokine levels. Our findings that the inflammation markers, TNFα and IL-6, increased in R6/2 mice and that these were normal in R6/2 mice treated with P110 (FIG. 4, B and C) suggest that these could be biomarkers for a trial of therapeutic intervention in HD patients.

Although much of the HD research focuses on pathologies associated with the CNS, it is clear that peripheral tissues are also affected in the disease. Importantly, a recent large GWAS (Genome-Wide Association Study) analysis of ˜4000 HD patients identifies genetic variations to explain the age of neurological symptoms onset in HD patients that differ from the predicted age of onset, based on the size of the CAG repeat. Fourteen significant pathways clustered by gene membership into three groups of genes: The largest group includes genes related to DNA repair, the second relates to genes that affect mitochondrial organization, release of cytochrome c (indicative of mitochondrial damage) and mitochondrial fission, and the third—to oxi-reductase activity. The authors suggested that these HD disease modifying genes in humans identified validated therapeutic targets in humans.

R6/2 mice exhibit a fast progression of the disease and are thus well suited to experimental analysis. More slowly developing models of HD have also been developed. Given that R6/2 mice are an accepted animal model for HD and R6/2 mice benefited from treatment with P110, as did cells and neurons derived from HD patients, and at least one of the biomarkers (mtND2) is also altered in human HD patients, results presented herein are promising. The biomarkers that we have identified are useful as surrogate markers for treatment benefit. Our mouse study also suggests that changes in these biomarkers correlate with disease progression in each individual. Our work provides the basis for identification of biomarkers that could be used as surrogate markers to determine the benefit of therapeutic intervention in diagnosed but asymptomatic HD patients to prevent or delay the onset of the disease.

### Materials and Methods

Peptide Treatment in Mouse Model:

All the experiments were in accordance with protocols approved by the Institutional Animal Care and Use Committee of Stanford University and were performed based on the National Institutes of Health Guide for the Care and Use of Laboratory Animals. Hemizygous R6/2 HD mice and their wild-type littermates (WT) were purchased from Jackson Laboratories and shipped to us at 5 weeks of age. The animals used in the P110 treatment study were implanted with a 28-day osmotic pump (Alzet, Cupertino Calif.) containing TAT₄₇₋₅₇ carrier control peptide or P110-TAT₄₇₋₅₇ (P110 peptide), which delivered to the mice at a rate of 3 mg/Kg/day, as described before. The first pump was implanted at 5 weeks of age and replaced once, after 4 weeks. For the intermittent treatment study, TAT or P110 were delivered as above, using a 1-week pump. After 3 weeks with no treatment, a new pump was implanted for another week of treatment, and mice were sacrificed three weeks later, at the age of 13 weeks.

Animal Survival and Behavior Study:

The overall survival during the study period was recorded and the remaining mice were sacrificed when they reached 13 weeks. All the behavior, survival tests and analyses were conducted by an experimenter who was blind to genotypes and drug groups.

Blood Collection and Measurement of Mitochondrial DNA Levels from Plasma and CSF:

Mouse blood was collected by retro-orbital bleeding. For the time course experiment, 200 μl of blood samples were collected from alternate eyes every 2 weeks from the age of 5 weeks to 13 weeks. In the P110 treatment study, 500 μl blood was collected at 13 weeks, just before euthanasia. Plasma was obtained by a single centrifugation step at 1600 g for 10 minutes, as previously reported. 100 μl of plasma samples were used to extract DNA, eluted in 60 μl elution buffer using Qiagen viral DNA kit (Qiagen). 1:10 DNA dilution was used in real-time PCR reaction.

Cerebrospinal fluids from three control and three HD subjects were obtained from the Human Brain and Spinal Fluid Resource Center, VA West Los Angeles Healthcare center, Los Angeles, Calif. 90073. Mitochondrial DNA from 200 μl CSF was extracted as above, using Qiagen viral DNA kit to minimize contamination of molecules present in CSF that inhibit the detection of DNA by PCR. 5 μl of undiluted CSF DNA was used in real-time PCR reaction. Human plasma samples were generously obtained from Dr. Leavitt from University of British Columbia; 6 plasma samples were from control subjects, 6 from presymptomatic HD subjects (mtHtt gene carrier), and 6 from affected HD patients. Each group included 3 male and 3 female subjects. DNA was extracted as above, and 1:2 dilution was used in real-time PCR.

RNA Extraction from Brain Tissue:

100 mg of brain tissue was used for RNA isolation, using RNAquaeous kit (Ambion) as manufacture protocol. 1 μg total RNA was used for the synthesis of first strand cDNA using PrimeScript 1ₛₜ strand cDNA synthesis kit (Takara) and 15 ng cDNA was used as a template for real-time PCR reaction.

Quantitative Analysis of DNA in Plasma by Real-Time PCR:

5 μl of DNA from mouse plasma, human plasma, human CSF (at respective dilution) or mouse brain tissue (15 ng) were used as templates for real-time PCR analysis. For assessment of nuclear DNA present in the samples and normalization of the measurements, we used the GAPDH—a housekeeping gene; (18S ribosomal DNA was used as well, yielding similar results). For the mouse GAPDH gene, we used forward SEQ ID NO:1 5′-GGACCTCATGGCCTACATGG-3′ and reverse SEQ ID NO:2 5′-TAGGGCCTCTCTTGCTCA-3′ primers. For the human GAPDH gene, we used forward SEQ ID NO:3, 5′-GTCGGAGTCAACGGATTTG-3′ and reverse SEQ ID NO:4, 5′-CCATGTAGTTGAGGTCAATGAA-3′. To detect circulating mitochondrial DNA, we used mouse mtND2 gene with forward SEQ ID NO:5, 5′-AACCCACGATCAACTGAAGC-3′ and reverse SEQ ID NO:6, 5′-TTGAGGCTGTTGCTTGTGTG-3′; for human mtND2, we used forward 5′-SEQ ID NO:7, CTATCTCGCACCTGAAAC-3′ and reverse SEQ ID NO:8, 5′-GAGGGTGGATGGAATTAAG-3′. PCR was performed using ABI/Life Technologies StepOnePlus real-time PCR instrument (Applied Biosystems) in a total volume of 20 containing 5 μl plasma DNA or 5 μl of 15 ng brain DNA, 10 μl Fast Sybr green master mix (Applied Biosystems), 1 μl primers (forward+reverse) at 2 μM using cycles as followed: 95° C. 20 sec, and 40 cycles of 95° C. 3 sec and 57° C. 30 sec followed by melt curve at 95° C. 15 sec, 60° C. 1 min and 95° C. 15 sec. We optimized the reaction using several primers for each of target genes according to the melting curve of respective primers in the assay. Relative changes in gene expression was calculated using 2^{-ΔΔC}T method where ΔΔC_{T}=(C_{T,mtND2}−C_{T,GAPDH})ₜᵣₑₐₜₘₑₙₜ−(C_{T,mtND2}−C_{T,GAPDH}) control. For treated samples, evaluation of 2^{-ΔΔC}T indicates the fold changes in gene expression relative to untreated control.

Measurement of DNA Damage (8-OHdG) by ELISA:

Urine was collected from WT and R6/2 mice at 13 weeks of age after 8 weeks of treatment with TAT control or P110. Urine, diluted 1:100 in water, was assayed as described in the manufacture protocol (Cell Biolabs) with a competitive ELISA assay kit for quantitative measurement of 8-OHdG. Urine samples were analyzed in parallel for creatinine content for normalization of 8-OHdG results according to the manufacture protocol (Cell Biolabs). The results are expressed as ng/ml 8-OHdG/μmol/L creatinine levels.

TNFα and IL-6 Measurements:

Plasma TNF-α and IL-6 levels were determined by a mouse TNF-α and IL-6 ELISA kit according to manufacturer's protocol (eBioscience, San Diego, Calif., USA) using 20 μl plasma collected and prepared as mentioned above.

Immunohistochemistry in Tissue Section s:

13 weeks old mice were sacrificed and skeletal muscles (quadriceps and hamstrings) and skin from the top dorsal area after hair removal were fixed in 4% paraformaldehyde in 0.1 M phosphate buffer pH 7.4. Tissues were processed for paraffin embedment and sections were used for immunohistochemical staining of mtHtt (EM-48, 1:200; Millipore) and 4-HNE staining (1:200; Abcam) using the IHC Select HRP/DAB kit (Millipore). The images were viewed using a Leica microscope with a 20× objective.

Statistical Analysis:

The results are presented as mean±SE. Statistical analysis was assessed by unpaired Student's t test and 1-way ANOVA using GraphPad Prism (GraphPad Software, Inc, La Jolla, Calif.). The standard Mantel Cox log-rank test was used to assess survival. Repeated measures two-way ANOVA with Bonferroni post hoc test was used for evaluation of the parameters in rotating rod test and DMP-DM. All tests were performed in a blinded way. p<0.05 was considered statistically significant.

Animal Model and Behavioral Tests:

Two cohorts of male B6CBA-Tg (HDexon1)62Gpb/3J (R6/2) mice and their wildtype (WT) littermates from Jackson laboratory were used for behavioral phenotyping (JAX Stock#006494). Cohort 1 mice consisted of n=10 WT and n=10 R6/2 mice and were housed under 12 hours light/dark cycle (7:00 am light on-7:00 pm light off). Cohort 1 mice were tested in Rotating Rod Test, Y-maze Spontaneous Alternation Test, and Delay Match-to-Place Dry Maze Test during the light on cycle of the day. Cohort 2 mice consisted of n=10 WT and n=10 R6/2 mice and were housed under 12 hours light/dark cycle (8:30 am light off-8:30 pm light on). Cohort 2 mice were tested in Social Discrimination Test using PhenoLab, Passive Avoidance Test, and Fear Conditioning Test during the light off cycle of the day. The mice were group housed 3-5 per cage and handled by experimenter for 5 days prior to the behavioral experiment. In addition to having ad libitum access to regular food and water, wet chow on disposable weight boat were provided at the bottom of the cage. Due to R6/2 mice having high sensitivity to vibration and noise, cages were hand-carried by the experimenters and mice were habituated on a cart outside or inside the testing rooms one hour prior to the tests. The experimenters were not aware of the genotype of the mice during the experiments. All behavioral procedures were conducted in accordance with protocols approved by the Institutional Animal Care and Use Committee of Stanford University, and were performed based on the National Institutes of Health Guide for the Care and Use of Laboratory Animals. All actions were considered for reducing discomfort of the animals throughout the study.

Rotating Rod Test:

Mice motor learning and coordination were accessed using Five Station Rota-Rod Treadmill (Med Associates Inc., St. Albans, Vt. Model ENV-575M) during 7, 9, and 11 weeks of age. Two days prior to the first testing at 7 weeks old, each mouse received 3 training trials. Each training trial was 60 s long at a fixed speed of 32 round per minute (rpm) with 5-10 minutes Inter-trial-intervals (ITIs). During the testing, each mouse received 2-3 trials of 4-40 rpm accelerated speed. Maximum duration of the each trial was 300 s with 15-20 min of ITIs. Mice were tested for a minimum of 2 trials per day, but removed from Rota-Rod and tested for a third trial if the following exclusion criteria were met—a mouse held on the rod instead of walking on it for two consecutive revolutions, or three cumulative revolutions during the trial, jumped off the rod instead of dropping off the rod due to lack of balance, or fell off the rod in less than 5 s. Average latency of the two trials to fall off the Rota-Rod or the latency when the mice met the exclusion criteria were used for data analysis. The Rota-Rod was cleaned with 10% alcohol between trials. A total of 19 mice (n=10 WT and n=9 R6/2) were used in the test.

Y-Maze Spontaneous Alternation Test:

Spontaneous alternations in mice were measured in a custom built Y-maze when the mice were 8 weeks old. The maze was made of opaque white plastic and had 3 equal arms of 40 cm length, 8 cm width, and 15 cm height. Each arm was labeled with a letter A, B, or C. Mice were placed in the maze facing arms B and C. The first entry was excluded from data analysis due to the fact that the animals were led to this initial arm. The total number of entries and sequence of entries into the arms were recorded for 8 minutes. Entries into the arms were defined as when all four paws entered into a new arm of the maze, and not when the mice moved to the center and returned to the same arm. The percentage of spontaneous alternation was calculated. Briefly, the experimenter analyzed the sequence of the arm entries A, B and C in a set of 3 entries or a triad. Every triad with all 3 letters was considered as alternation (e.g. ABC, BCA, CAB) and percent spontaneous alternation was calculated using the number of alternation divided by the total possible triads times 100. For example “ABCACAB”. The data was then broken into triads of entries, a sequence with repeating letters such as “ABA” or CAC” would be scored as a non-alternation while a sequence with all three letters, e.g. “ABC” or “CBA” would be scored as an alternation. For our sample above the first triad was “ABC”, the second was “BCA”; however the third triad “CAC” would not be alternation. In this sample data, there were total of 7 entries, 5 possible triads, and 3 alternations. Percent spontaneous alternation would be (⅗)*100=60%. The Y-maze was cleaned with 10% alcohol between each mouse. A total of 19 mice (n=10 WT and n=9 R6/2) were used in this test.

Delay Match-to-Place Dry Maze:

The Delay Match-to-Place Dry Maze (DMP-DM) test was conducted using a custom built circular shaped platform 122 cm in diameter with 40 holes elevated 50 cm from the floor. The test consisted of 7 days of testing when the mice were 10 weeks old. Each hole was 5 cm in diameter and an escape tube filled with bedding was attached to only one of the holes. The hole with an escape tube was defined as the Target Escape Hole (TEH). Remaining 39 holes without the escape tube were covered with a piece of plastic so the mice would not accidentally drop into the holes. A short lip was placed around the edge of the maze to prevent the animals from falling off the platform. High overhead lighting with 900 lux was used to create an aversive stimulus that would encourage the animals to seek out the Target Hole to escape from the light. The maze was surrounded by privacy blinds and distinct visual cues were placed on the privacy blinds. An individual mouse was given a series of 4 trials per day to find the escape hole with 10-12 min ITIs. Maximum duration for each trial was 90 s. The bright lights in the testing room were kept dim prior to the start of a trial. The subject mouse was placed under an opaque box in the pseudo-randomized positions around the edge of the maze. The experimenter turned on the bright light after 10 s and the box was removed to allow the mouse to find TEH. If the mouse found and entered into the TEH before 90 seconds, the experiment was stopped. Mice that could not find the TEH or enter the escape tube were led to it by the experimenter and allowed to enter. Mice were allowed to remain in the tube for 10 seconds after each trial and returned to the home cage. After each trial, the apparatus was cleaned with 10% alcohol to eliminate odor cues. At the start of day 2-7, the location of the TEH was moved to a new escape hole while everything else remained the same. Mice were tracked with Ethovision XT (Noldus Information Technology, Wageningen, Netherlands) and latency to find the TEH, distance moved, and velocity were recorded. A total of 19 mice (n=10 WT and n=9 R6/2) were used in the test.

Social Discrimination Test Using PhenoLab:

The PhenoLab cages were custom built cages made of acrylic plastic with 30 cm length×30 cm width×60 cm height. Mice were individually housed and habituated in the cages for 4 days prior to social discrimination test. Infrared cameras were mounted on top of the cages to monitor the mice inside the cages. The mice were 6 weeks old when they were introduced into the cages. All n=10 WT and n=10 R6/2 mice were tested simultaneously in 20 individual PhenoLab cages. Each cage was equipped with a food tray, water bottle, running wheel (Med Associates Inc., St. Albans, Vt. Model ENV-044) and shelter box (red transparent polycarbonate). Mice had ad libitum access to all enrichments and were not disrupted by the experimenter during the habituation. In the subsequent social discrimination test, the running wheel was removed and two identical stainless steel pencil cups (11 cm height×10 cm diameter solid bottom; with stainless steel bars spaced 1 cm apart) were inverted and placed in two corners of the cage adjacent from one another. A novel object (plastic cap) and a novel young juvenile mouse: Stranger 1 were placed under each cup and the subject mouse was allowed to explore for 2 hours. After 2 hours, the Stranger 1 and the cup were repositioned to the corner where the novel object was located. The novel object was removed from the cage and a second novel young juvenile mouse: Stranger 2 was placed under the cup. Subject mice were allowed to explore the Stranger 1 and Stranger 2 mice for 10 min after Stranger 2 was introduced into the cage. Both juvenile mice were 5 weeks old C57Bl/6J male mice (JAX stock#000664) and they were housed in different cages. Subject mice were tracked with Ethovision XT (Noldus Information Technology, Wageningen, Netherlands) and center of the subject mice within 4 cm virtual zones around the cups were used as interaction time. A total of 19 mice (n=19 WT and n=10 R6/2) were used in the test.

Passive Avoidance Test:

The Passive Avoidance Test (PAT) was conducted using GIMINI avoidance system (San Diego Instruments, San Diego, Calif.) when the mice were 8 weeks old. This automated system contained two compartments which were separated by a guillotine door (gate). Both compartments had grid floor which could deliver electric shock, but one compartment was lighted while the other was dark. The experiment consisted of 1 day of habituation, 1 day of training, and 2 days of testing. On habituation day, the mouse was placed in the lighted compartment. After 30 s acclimation, the gate was opened and the mouse was allowed to explore both compartments freely. The gate was programmed to close when the mouse entered the dark compartment to prevent the mouse from returning to the lighted compartment. The mouse was removed from the system and returned to home cage after it entered the dark compartment. On the following day, Training Day, the mouse was placed in the light compartment. After 30 s of acclimation the gate was opened and the mouse was allowed to explore both compartments freely. The gate was closed after it entered the dark compartment. 3 s after the gate was closed, an electric shock (0.5 mA for 2 seconds) was delivered. The mouse remained in the dark compartment for additional 30 s before being removed and returned to the home cage. On the following day, Day 1 Testing Day, the mouse was placed in the lighted compartment. After 5 seconds acclimation, the gate was opened. When the mouse entered the dark compartment, the gate was closed and trial ended. Mouse was returned to the home cage. Seven days after training, Day 7 Testing Day, same procedure were repeated as Day 1 Testing Day. Maximum duration of each trial was 300 s after the gate was opened. The time between the gate opening and the mouse passing through the gate was recorded as latency time. The compartments were cleaned with 1% Virkon between each animal. A total of 19 mice (n=9 WT and n=10 R6/2) were used in the test.

Fear Conditioning Test:

The Fear Conditioning Test (FCT) was conducted using Coulbourn Instruments fear conditioning chambers (Whitehall, Pa.) when the mice were 11-12 weeks old. The test consisted of Day 1 Training, Day 2 Cued Testing, and Day 3 Contextual Testing. During Day 1 Training and Day 3 Contextual Testing, mice were tested in distinct Context A (metal grid floor, square shape clear chamber, yellow dim light, mint extract as odor cue, and 10% simple green solution to clean the chamber between each mouse). During Day 2 Cued Testing mice were tested in Context B (plastic floor, round shape opaque chamber, blue dim light, vanilla extract, 70% alcohol to clean the chamber between each mouse, and different testing room). On Day 1 Training, the mice were acclimated in the chamber for 200 s followed by 5× pairing of tones and shocks. The tones were 20 s duration, 2 kHz frequency and 70 dB loud. The shocks were 2 s duration at 0.5 mA shock intensity. The time between a tone and a shock pairing was 18 s, and the ITIs between the tones were 100 s. The mice were removed from the chamber and returned to the home cage 80 s after the last tone. On Day 2 Cued Testing, the mice were acclimated in the chamber for 200 s followed by 3 tones without any shock. The tones were 20 s duration, 2 kHz frequency and 70 dB loud. The ITIs between each tones were 100 s. The mice were removed from the chamber and returned to the home cage 80 s after the last tone. On Day 3 Contextual Testing, the mice were placed in Context A testing chamber for 5 min without any tone or shock. Mice were returned to the home cage after the trial. The mice freezing behavior was recorded with a camera above the chamber and freezing was defined as the complete lack of motion for a minimum of 0.75 s, as assessed by FreezeFrame software (Actimetrics, Evanston, Ill.). A total of 15 mice (n=9 WT and n=6 R6/2) were used in the test.

Statistical Analysis:

Statistical analysis were processed using GraphPad Prism software version 5 (GraphPad Software, Inc, La Jolla, Calif.). Data were presented as mean±SEM and statistically significant was defined as P<0.05. Repeated measures two-way ANOVA with Bonferroni post hoc test was used for evaluation of the parameters in Rotating Rod Test and DMP-DM. Unpaired student's t-test was used for Y-maze total entries, PAT and FCT. One-sample t-test was used for spontaneous alternation comparing the mean alternation of each genotype to the hypothetical value of 50%. Paired t-test was used to compare the time spent in Stranger 1 vs Novel Object during the sociability session for both genotypes. Wilcoxon nonparametric paired t-test was used in social novelty session for the WT mice and paired t-test was used for the R6/2 mice when comparing the time spent in Stranger 1 and Stranger 2 zones during Social Discrimination Test. D'Agostino and Pearson omnibus normality test was used to determine the normal distribution of data set. Kolmogorov-Smirnov test was used to determine the normal distribution of the data set for Fear Conditioning since number of R6/2 mice were too small for D'Agostino and Pearson omnibus normality test.

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

1. A method for assessing the efficacy of a therapeutic agent or therapeutic regimen in the treatment of a subject with a neurodegenerative disorder, the method comprising:
identifying the subject having the neurodegenerative disorder, wherein the subject is administered the therapeutic agent or therapeutic regimen;
isolating at least two samples of peripheral non-CNS tissue or cerebrospinal fluid (CSF) from the subject, wherein a first sample of the at least two samples is isolated before administering the therapeutic agent or therapeutic regimen and a second sample of the at least two samples is isolated after administering the therapeutic agent or therapeutic regimen; quantitating the presence of at least one peripheral biomarker in the first and second samples isolated from the subject, wherein the peripheral biomarker is (i) a marker of mitochondrial and cell integrity; (ii) mtHtt aggregation in peripheral tissue; or (iii) a marker of increased oxidative stress to determine a first level in the first sample and a second level in the second sample of the at least one peripheral biomarker; and
comparing the first and second levels to identify a change in relative levels of the at least one peripheral biomarker, wherein identification of the change in relative levels of the at least one peripheral biomarker is indicative of the efficacy of the therapeutic agent or regimen.

2. The method of claim 1, wherein the peripheral non-CNS tissue is selected from peripheral blood, plasma, serum, urine, skin, and muscle.

3. The method of claim 1, wherein the neurodegenerative disorder is Huntington's Disease (HD), Parkinson's Disease or Alzheimer's Disease.

4. The method of claim 3, wherein the subject is a human and the neurodegenerative disorder is HD.

5. The method of claim 3, wherein the subject has a genetic disposition to HD.

6. The method of claim 3, wherein the subject is an animal in a pre-clinical model of HD.

7. The method of claim 1, wherein the therapeutic agent inhibits mitochondrial fission.

8. The method of claim 6, wherein the therapeutic agent comprises P110 peptide.

9. The method of claim 8, wherein the therapeutic agent comprises a peptide comprising (i) YGRKKRRQRRR (SEQ ID NO: XX), (ii) GG, and (iii) DLLPRGS (SEQ ID NO: YY) attached in order (i), (ii), and (iii) from amino terminus to carboxyl terminus.

10. The method of claim 8, wherein the therapeutic agent comprises a peptide consisting of (i) YGRKKRRQRRR (SEQ ID NO: 9), (ii) GG and (iii) DLLPRGS (SEQ ID NO: 10) attached in order (i), (ii), and (iii) from amino terminus to carboxyl terminus; or a pharmaceutically acceptable salt thereof.

11. The method of claim 1, wherein the at least two samples are selected from the group consisting of plasma, urine, skin and muscle tissue.

12. The method of claim 11, wherein levels of mitochondrial DNA (mtDNA) are measured.

13. The method of claim 12, wherein the mtDNA comprises a sequence encoding sequences encoding NADH dehydrogenase; ATP synthase; cytochrome c oxidase; or ubiquinol cytochrome c reductase.

14. The method of claim 12, wherein measuring is performed by quantitative PCR.

15. (canceled)

16. The method of claim 11, wherein levels of 8-OHdG or of 4-HNE adducts are measured.

17. The method of claim 16, wherein measuring is performed by ELISA or wherein levels of mtHtt aggregation are measured.

18-21. (canceled)

22. The method of claim 1, wherein an efficacious therapy normalizes levels of the at least one peripheral biomarker in the second sample to a level substantially the same as a normal control.

23. The method of claim 22, comprising continuing treatment of the subject with a therapeutic agent or regimen determined to be efficacious.

24. The method of claim 22, comprising discontinuing treatment of the subject with a therapeutic agent or regimen determined not to be efficacious.

25-27. (canceled)

28. A method for identifying a time to initiate a therapeutic intervention in an individual predisposed to develop a neurodegenerative disorder, the method comprising:
quantitating the presence of at least one peripheral biomarker selected from (i) a marker of mitochondrial and cell integrity; (ii) mtHtt aggregation in the peripheral tissue; and (iii) a marker of increased oxidative stress; in at least two patient samples obtained at two or more time points to obtain a result, where the disease status of the individual is expected to differ between the time points as the result of administering a therapeutic agent or therapeutic regimen; and
identifying a time to initiate a therapeutic intervention in an individual predisposed to develop a neurodegenerative disorder based on the result.

29. A method for treating Huntington's Disease (HD) in a mammal, the method comprising administering a therapeutic agent comprising P110 peptide to the mammal, wherein the therapeutic agent comprising P110 peptide is administered in accordance with an intermittent dosing regimen whereby treatment periods are interrupted by rest periods wherein the therapeutic agent comprising P110 peptide is not administered to the mammal.

30. The method of claim 29, wherein the therapeutic agent comprises a peptide comprising (i) YGRKKRRQRRR (SEQ ID NO: 9), (ii) GG, and (iii) DLLPRGS (SEQ ID NO:10) attached in order (i), (ii), and (iii) from amino terminus to carboxyl terminus.

31. The method of claim 29, wherein the therapeutic agent comprises a peptide consisting of (i) YGRKKRRQRRR (SEQ ID NO: 9), (ii) GG and (iii) DLLPRGS (SEQ ID NO: 10) attached in order (i), (ii), and (iii) from amino terminus to carboxyl terminus; or a pharmaceutically acceptable salt thereof.

32. The method of claim 29, wherein the mammal is a human.

33. The method of claim 29, further comprising repetitive cycles of the intermittent dosing regimen.

34. The method of claim 29, wherein the mammal has an expanded trinucleotide CAG repeat in its gene encoding the huntingtin protein.

35. A method for predicting onset of Huntington's Disease (HD) in a mammal, the method comprising:
a) isolating peripheral tissue from the mammal, wherein the peripheral tissue is isolated from non-central nervous system tissue of the mammal;
b) quantitating the presence of at least one peripheral biomarker in the peripheral tissue isolated from the subject, wherein the peripheral biomarker is (i) a marker of mitochondrial and cell integrity; (ii) mtHtt aggregation in peripheral tissue; or (iii) a marker of increased oxidative stress to determine a level of the at least one peripheral biomarker in the peripheral tissue; and
c) comparing the level of the at least one peripheral biomarker in the peripheral tissue to that of a normal control, wherein a difference in the level of the at least one peripheral biomarker in the peripheral tissue isolated from the subject relative to that of the normal control is positively correlated with onset of HD in the mammal.

36. The method of claim 35, further comprising treating a mammal predicted to be susceptible to onset of HD.
